(19) United States
(12) Patent Application Publication
(10) Pub. No.: US 2026/0224761 A1
(43) Pub. Date: Aug. 6, 2026

(54) DUAL LABELLED COMPOUNDS TARGETING THE PROSTATE SPECIFIC MEMBRANE ANTIGEN

(71) Applicant: CENTRE HOSPITALIER UNIVERSITAIRE VAUDOIS (CHUV), Lausanne (CH)

(72) Inventors: Margret SCHOTTELIUS, Lausanne (CH); Fijs W.B. VAN LEEUWEN, Noordwijkerhout (NL); Tessa BUCKLE, Zoeterwoude (NL); Hans-Jürgen WESTER, Schweitenkirchen (DE)

(21) Appl. No.: 19/148,499

(22) PCT Filed: Jan. 17, 2024
(86) PCT No.: PCT/EP2024/051083

§ 371 Date: Jul. 16, 2025

(30) Foreign Application Priority Data
Jan. 17, 2023 (EP) 23151995.0

(51) Int. Cl. A61K 51/08; A61K 49/00; C12Q 1/34; G01N 21/64

(52) U.S. Cl. CPC A61K 51/088; A61K 49/0032; A61K 49/0056; C12Q 1/34; G01N 21/6458; A61K 2123/00; G01N 2333/948

(57) ABSTRACT

The present invention relates to a compound of formula (I) or a pharmaceutically acceptable salt thereof: (I) wherein A is a group comprising a chelator group and a fluorescent dye. The invention provides, inter alia, radiolabeled and/or fluorescent hybrid probes for PSMA-targeted radio-guided and/or fluorescence-guided surgery. The invention further provides compounds and compositions for imaging purposes, in particular for imaging solid cancers.

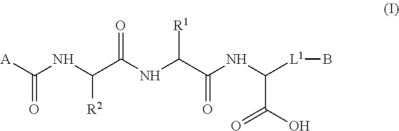

Pioneering studies on hybrid (nuclear/fluorescent) intraoperative guidance during sentinel lymph (SN) node procedures using ICG-[^{99m}Tc]nanocolloid (van der Poel, H. G. et al. Eur. Urol., 2011, 60, 826-833), a dual-labelled (hybrid) tracer consisting of the fluorescent dye indocyanine green (ICG) and [^{99m}Tc]nanocolloid, have shown improvements in intraoperative (optical) SN detection over [^{99m}Tc]nanocolloid alone or in combination with blue dye (Dell'Oglio, P. et al., Eur. Urol., 2020, 78, 865-872). The development and progressive maturation of intraoperative detection devices such as the DROP-IN Gamma probe (Dell'Oglio, P. et al., Eur. Urol., 2021, 79, 124-132; Meershoek, P. et al., Eur. J. Nucl. Med. Mol. Imaging, 2019, 46, 49-53) or the da Vinci Xi firefly fluorescence laparoscope (Meershoek, P. et al., J. Robot. Surg., 2021, 15, 751-760) for robotic surgery have further boosted the interest in the development and clinical application of targeted probes for combined radio- and fluorescence guided (robotic) surgery.

In this context, particular focus has been directed towards prostate cancer (PC) because of the particularly high expression of prostate specific membrane antigen (PSMA) on more than 90% of primary and recurrent PCs (Silver, D. A. et al., Clin. Cancer Res., 1997, 3, 81-85). The development of specific radiolabeled, fluorescent and hybrid probes for PSMA-targeted radio-guided or fluorescence-guided surgery (RGS or FGS, respectively) and hybrid surgical guidance, respectively, has been greatly facilitated by the accessibility of a variety of “lead structures” in the form of clinically established PSMA-targeted imaging agents for positron emission tomography (PET) and single photon emission computed tomography (SPECT). Many fluorescent and hybrid PSMA-probes are structurally based on already-optimized radiopharmaceutical designs, and the results of these diverse efforts have been summarized in several comprehensive reviews (Hensbergen, A. W. et al., Bioconjug. Chem., 2020, 31, 375-395; D E Vries, H. M., Q J. Nucl. Med. Mol. Imaging, 2021, 65, 261-270; Derks, Y. H. W. et al., Theranostics, 2019, 9, 6824-6839; Maurer, T. et al., J. Nucl. Med., 2019, 60, 156-157).

Amongst the prior art compounds developed for RGS, notable are [¹¹¹In]PSMA-I&T, a gamma-emitting PSMA-probe (Schottelius, M. et al., EJNMMI Res., 2015, 5:68; Rauscher, I. et al., BJU Int., 2017, 120, 40-47) and [⁶⁸Ga]PSMA-I&F (Schottelius, M. et al., J. Nucl. Med., 2019, 60, 71-78). These compounds however have several limitations. First, the use of a short-lived positron emitter such as ⁶⁸Ga is not compatible with the clinical workflow of preoperative imaging and subsequent RGS/FGS, usually involving tracer injection on the evening prior to surgery, followed by preoperative imaging and surgery the next morning (Maurer, T. et al., Eur. Urol., 2019, 75, 659-666). More importantly, the precise spatial detection of the 511 keV gamma rays from positron annihilation in the surgical field requires heavily collimated surgical probes and is thus not practicable. Lastly, due the relatively high hydrophilicity of [⁶⁸Ga]PSMA-I&F, the tracer displays fast clearance kinetics and thus sub-optimal clearance characteristics for achieving sustained tracer delivery for maximal tumor uptake. The use of the longer-lived gamma emitter ¹¹¹In for labeling is also not viable as it is associated with high cost and thus, not clinically practicable. Technetium-99m (^{99m}Tc) has been explored due to its improved nuclear properties, including a physical half-life of 6 hrs, biological half-life of around 1 day and it is readily detectable gamma rays with a photon energy of 140 KeV (which have a comparable wavelength to those emitted by conventional X-ray diagnostic equipment). [^{99m}Tc]PSMA-I&S was developed with these properties in mind (Robu, S. et al., J. Nucl. Med., 2017, 58, 235-242), however this compound also displayed fast clearance kinetics and thus suboptimal clearance characteristics for achieving sustained tracer delivery for maximal tumor uptake.

In view of the problems in the prior art, it was an object of the present invention to provide a hybrid PSMA-targeted imaging probe with more efficient tumor targeting via a prolonged circulation half-life, which results in an increase in accumulation in PSMA-expressing tumors. A further object of the present invention was to provide PSMA-targeted imaging probes with improved signal/background ratios, at late time points (e.g., 4-21 h post injection (p.i.)) and in particular for sensitive intraoperative lesion detection.

Thus, the technical problem underlying the present invention is to provide an improved hybrid PSMA-targeted imaging probe, in particular for application in hybrid surgical guidance.

The solution to this problem is provided by the embodiments as defined herein below and as characterized by the claims.

The invention, accordingly, relates to the following:
1. A compound of formula (I) or a pharmaceutically acceptable salt thereof:

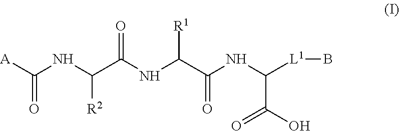

wherein
A is a group comprising a chelator group and a fluorescent dye; R¹ is an —(C₀₋₆ alkylene)-(optionally substituted bicyclic aryl) or —(C₀₋₆ alkylene)-(optionally substituted bicyclic heteroaryl), wherein the aryl in said —(C₀₋₆ alkylene)-(optionally substituted bicyclic aryl) and the heteroaryl in said —(C₀₋₆ alkylene)-(optionally substituted bicyclic heteroaryl) are each optionally substituted with one or more groups R³;
R² is an —(C₀₋₆ alkylene)-(optionally substituted monocyclic aryl), —(C₀₋₆ alkylene)-(optionally substituted bicyclic aryl), —(C₀₋₆ alkylene)-(optionally substituted monocyclic heteroaryl), or —(C₀₋₆ alkylene)-((optionally substituted bicyclic heteroaryl), wherein the aryl in said —(C₀₋₆ alkylene)-(optionally substituted monocyclic aryl) and in said —(C₀₋₆ alkylene)-(optionally substituted bicyclic aryl) and the heteroaryl in said —(C₀₋₆ alkylene)-(optionally substituted monocyclic heteroaryl) and in said —(C₀₋₆ alkylene)-(optionally substituted bicyclic heteroaryl) are each optionally substituted with one or more groups R⁴;
each R³ is independently selected from halogen, C₁₋₆ alkyl, C₂₋₆ alkenyl, C₂₋₆ alkynyl, C₁₋₆ haloalkyl, —OR⁵, —SO₂R⁵, —NO₂, —CN, —C(O)R⁵, —C(O)OR⁵, —C(O)N(R⁵)₂, —NR⁵R⁵, —NCO, —NCS, —SR⁵, —N(R⁵)C(O)R⁵ and —N₂;
each R⁴ is independently selected from halogen, C₁₋₆ alkyl, C₂₋₆ alkenyl, C₂₋₆ alkynyl, C₁₋₆ haloalkyl, —OH, —OR⁵, —SO₂R⁵, —NO₂, —CN, —C(O)R⁵, —C(O)OR⁵, —C(O)N(R⁵)₂, NR⁵R⁵, —NCO, —NCS, —SR⁵, —NR⁵C(O)R⁵ and —N₂;
each R⁵ is independently selected from hydrogen, halogen, C₁₋₆ alkyl;
L¹ is a linear —(C₁₋₃₀ alkylene)- group, wherein one or more —CH₂— units in said alkylene are optionally replaced by a group independently selected from —O—, —NH—, —N(C₁₋₆ alkyl)-, —CHR⁶—, —C(R⁶)₂—, —CO—, —S—, —SO—, —SO₂— or —(C₃₋₆ cycloalkylene)-; wherein each R⁶ is independently selected from halogen, C₁₋₆ alkyl, C₂₋₆ alkenyl, C₂₋₆ alkynyl, C₁₋₆ haloalkyl, —OH, —O(C₁₋₆ alkyl), —C(O)C₁₋₆ alkyl, —CN, —NH₂, —NH(C₁₋₆ alkyl) and —N(C₁₋₆ alkyl)(C₁₋₆ alkyl);
and
B is selected from —EuK or —EuE;
wherein —EuK is

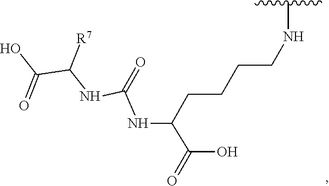

—EuE is

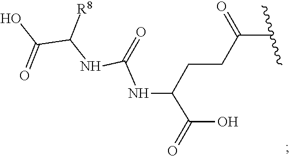

wherein R⁷ and R⁸ may be the same or different, wherein R⁷ and R⁸ are independently selected from —(C₁₋₆ alkylene)-C(O)OH, preferably wherein R⁷ and R⁸ are each —(C₂ alkylene)-C(O)OH;
wherein
the fluorescent dye is selected from Cy3, Cy3B, Cy3.5, Cy5, Cy5.5, Cy7, carbocyanine, indocarbocyanine, oxacarbocyanine, thiacarbocyanine, merocyanine, polymethine, coumarine, rhodamine, rhodamine B, xanthene, fluorescein, boron-dipyrromethane (BODIPY), VivoTag-680, Vivo Tag-S680, Vivo Tag-S750, AlexaFluor647, AlexaFluor660, AlexaFluor680, AlexaFluor700, AlexaFluor750, AlexaFluor790, Dy676, Dy677, Dy682, Dy752, Dy780, DyLight547, DyLight647, DyLight680, HiLyte Fluor 647, HiLyte Fluor 680, HiLyte Fluor 750, IRDye 8000W, IRDye 800RS, IRDye 700DX, ADS780WS, ADS830WS, ADS832WS and derivatives thereof.
2. The compound of formula (I) according to item 1, wherein the chelator group is selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]-hexadecan (D02A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphate) (DPDP), diethylenetriaminepentaacetic acid (DTPA), ethylenediamine-N,N′-tetraacetic acid (EDTA), ethylene glycol-0,0-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridin-bis(methyl-enamine)tetraacetic acid (TMT), 1,4,7,10-tetraazacyclo-tridecan-N,N′,N″,N′″-tetraacetic acid (TRITA), triethylenetetraaminehexaacetic acid (TTHA), N,N′-bis[(6-carboxy-2-pyridyl)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl}heptanedioic acid bis-[(3-hydroxy-1,6-dimethyl-4-oxo-1,4-dihydro-pyridin-2-ylmethyl)-amide](THP), mercaptoacetyl triserine (mas₃), mercaptoacetyl triglycine (mag₃) and derivatives thereof.
3. The compound of formula (I) according to item 1 or 2, wherein
   i) B is EuK having the configuration

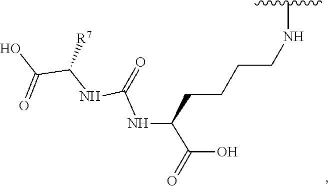

or EuE having the configuration

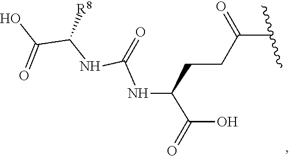

wherein R⁷ and R⁸ may be the same or different, wherein R⁷ and R⁸ are each independently selected from —(C₁₋₆ alkylene)-C(O)OH, preferably wherein R⁷ and R⁸ are each —(C₂ alkylene)-C(O)OH;
and/or
ii) wherein the chelator group is -mas₃ or a thioether or thioester derivative thereof, -mag₃ or a thioether or thioester derivative thereof, -DOTAGA or -DOTA, wherein
   -mas₃ or a thioether or thioester derivative of -mas₃ is selected from

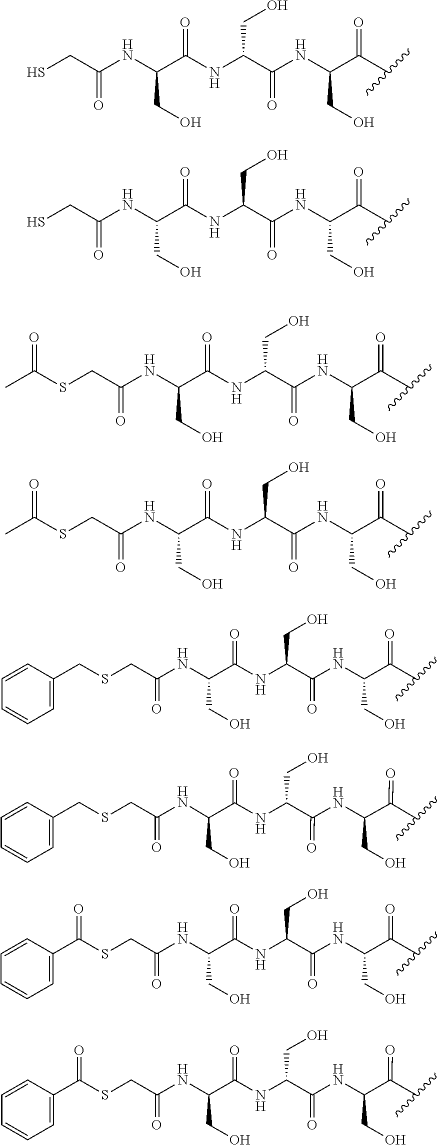

-mag₃ or a thioether or thioester derivative of -mag₃ is selected from

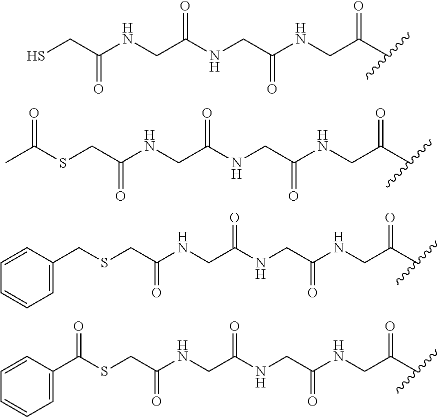 [SMILES: *C(=O)CNC(=O)CNC(=O)CNC(=O)CS ; *C(=O)CNC(=O)CNC(=O)CNC(=O)CSC(C)=O ; *C(=O)CNC(=O)CNC(=O)CNC(=O)CSCc1ccccc1 ; *C(=O)CNC(=O)CNC(=O)CNC(=O)CSC(=O)c1ccccc1]

-DOTAGA is

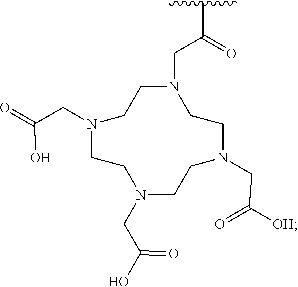 [SMILES: *C(=O)CN1CCN(CC(=O)O)CCN(CC(=O)O)CCN(CC(=O)O)CC1]

and/or
iii) wherein the fluorescent dye is Cy5, Cy 5.5, Cy7 or a derivative thereof.
4. The compound of formula (I) according to any one of items 1 to 3, wherein the fluorescent dye is a derivative of Cy5, Cy7 or derivatives thereof, preferably the fluorescent dye is

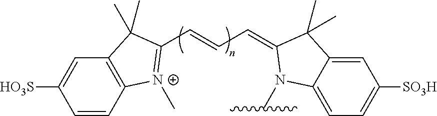

wherein n is 2 or 3.
5. The compound of formula (I) according to any one of items 1 to 4, wherein R¹ is a —(C₁ alkylene)-(optionally substituted bicyclic 10-membered aryl) and R² is a —(C₁ alkylene)-(optionally substituted monocyclic 6-membered aryl), wherein the bicyclic 10-membered aryl in said —(C₁ alkylene)-(optionally substituted bicyclic 10-membered aryl) is each optionally substituted with one or more groups R³, and the monocyclic 6-membered aryl in said —(C₁ alkylene)-(optionally substituted monocyclic 6-membered aryl) is substituted with an R⁴ group —OH, preferably having the configuration

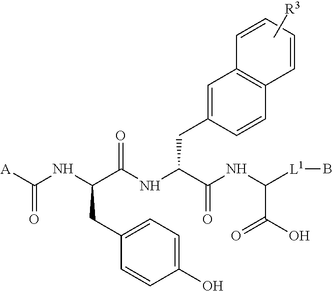

6. The compound of formula (I) according to any one of items 1 to 5, wherein the compound is

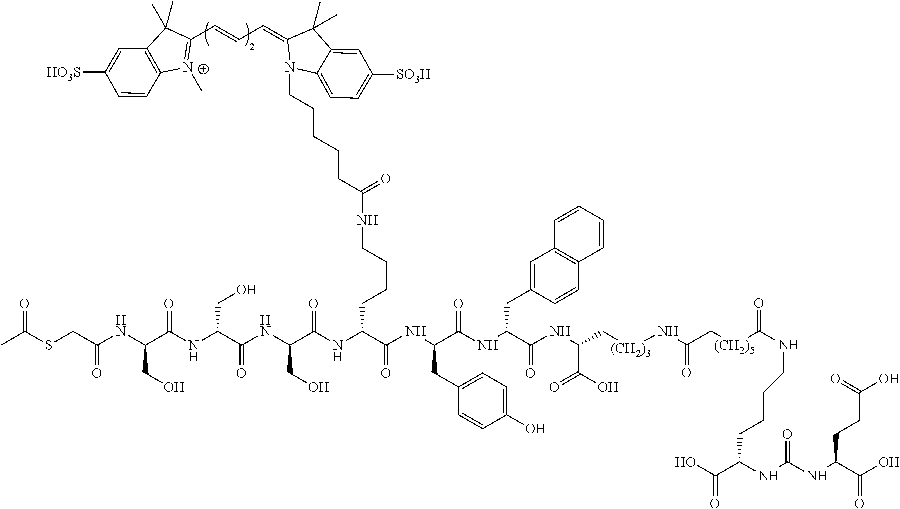

7. A composition comprising the compound of formula (I) according to any one of items 1 to 6 and a radionuclide, preferably wherein the radionuclide is selected from ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁷Ga, ⁶⁸Ga, ⁸⁹Zr, ⁸⁹Y, ⁹⁰Y, ^{94m}Tc, ^{99m}Tc, ⁹⁷Ru, ¹⁰⁵Rh, ¹⁰⁹Pd, ¹¹¹Ag, ¹¹⁰In, ¹¹¹In, ¹¹³In, ¹¹⁴In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁷Lu, ¹⁸⁶Re, ¹⁸⁸Re, ¹⁹¹Pt, ¹⁹⁷Hg, ¹⁹⁸Au, ¹⁹⁹Au, ²⁰³Pb, ²¹²Pb, ²¹¹At, ²¹²Bi, ²¹³Bi, ²²³Ra, ²²⁵Ac, and ²²⁷Th, and a cationic molecule comprising ¹⁸F, such as ¹⁸F—[AlF]²⁺.
8. The composition according to item 7, wherein the radionuclide is ^{99m}Tc.
9. The composition according to item 8, wherein the compound of formula (I) comprises a chelator group having at least 2, preferably 3, more preferably 4 functional groups for bonding to the radionuclide ^{99m}Tc, wherein said functional group comprises an atom having 1 to 3 free electron lone pair(s), wherein said atom is O, P, N or S; and
   wherein the chelator group is preferably bidentate, tridentate or tetradentate; and/or
   wherein the chelator group is cyclic or acyclic.
10. A pharmaceutical composition comprising the compound of formula (I) according to any one of items 1 to 6 or the composition of any one of items 7 to 9 and a pharmaceutically acceptable excipient.
11. A compound of formula (I) according to any one of items 1 to 6, the composition of any one of items 7 to 9 or the pharmaceutical composition of item 10 for use in diagnostics, preferably for use in imaging, more preferably for use in imaging a solid cancer.
12. The compound of formula (I), the composition or the pharmaceutical composition for use of item 11, wherein the solid cancer is selected from lung cancer, gastrointestinal cancer, colorectal cancer, colon cancer, anal cancer, liver cancer, pancreatic cancer, stomach cancer, genitourinary cancer, bladder cancer, biliary tract cancer, hepatobiliary cancer, testicular cancer, cervical cancer, ovarian cancer, uterine cancer, endometrial cancer, vaginal cancer, vulvar cancer, malignant mesothelioma, esophageal cancer, laryngeal cancer, prostate cancer, breast cancer, brain cancer, neuroblastoma, Ewing's sarcoma, osteogenic sarcoma, kidney cancer, epidermoid cancer, skin cancer, melanoma, head and/or neck cancer, mouth cancer, thymoma, Merkel-cell cancer, and neuroendocrine cancer, preferably wherein the solid cancer is prostate cancer.
13. The compound of formula (I), the composition or the pharmaceutical composition for use of any one of items 11 or 12, wherein the use comprises administering the compound of formula (I), the composition or the pharmaceutical composition to a subject and thereafter imaging the tumor.
14. The compound of formula (I), the composition or the pharmaceutical composition for use of any one of items 11 to 13, wherein the imaging comprises positron emission spectroscopy (PET), single photon emission computed tomography (SPECT), scintigraphy, (intraoperative)gamma-tracing/imaging, or (intraoperative)beta-tracing; and
   wherein the imaging comprises fluorescence imaging, optionally wherein the imaging comprises fluorescence spectroscopy.
15. An in vitro method of imaging tissues expressing or over expressing prostrate specific membrane antigen (PSMA), the method comprising contacting said tissue with the compound of formula (I) according to any one of items 1 to 6, the composition of any one of items 7 to 9 or the pharmaceutical composition of item 10.
16. A method of imaging tissues that are associated with pathogenic populations of cells expressing or over expressing prostrate specific membrane antigen (PSMA), the method comprising administering the compound of formula (I) according to any one of items 1 to 6, the composition of any one of items 7 to 9 or the pharmaceutical composition of item 10.
17. Use of the compound of formula (I) according to any one of items 1 to 6, the composition of any one of items 7 to 9 or the pharmaceutical composition of item 10 as an imaging agent.
18. Use of the compound of formula (I) according to any one of items 1 to 6, the composition of any one of items 7 to 9 or the pharmaceutical composition of item 10 as an analytical reference.
19. Use of the compound of formula (I) according to any one of items 1 to 6, the composition of any one of items 7 to 9 or the pharmaceutical composition of item 10 as an in vitro screening tool.
20. A pharmaceutical composition comprising the compound of formula (I) according to any one of items 1 to 6, the composition of any one of items 7 to 9 or the pharmaceutical composition of item 10 for use in therapy, preferably for use in the treatment or prevention of a solid cancer, more preferably for use in the treatment or prevention of prostate cancer.
21. Use of the compound of formula (I) according to any one of items 1 to 6, the composition of any one of items 7 to 9 or the pharmaceutical composition of item 10 for the manufacture of a medicament for the treatment or prevention of a disease, preferably wherein said disease is a solid cancer, more preferably wherein said disease is prostate cancer.
22. A method of treating or preventing a disease/disorder in a subject, wherein the disease/disorder is a solid cancer, the method comprising administering the compound of formula (I) according to any one of claims 1 to 6, the composition of any one of claims 7 to 9 or the pharmaceutical composition of claim 10 to a subject in need thereof.
23. Use of a compound of formula (I) according to any one of claims 1 to 6, the composition of any one of claims 7 to 9 or the pharmaceutical composition of claim 10 for the preparation of an agent for diagnosing a disorder/disease.
24. A method of delivering a compound of formula (I) according to any one of claims 1 to 6, the composition of any one of claims 7 to 9 or the pharmaceutical composition of claim 10, the method comprising parenteral administration of said compound, composition or pharmaceutical composition.
25. A kit comprising a compound of formula (I) according to any one of claims 1 to 6 and one or more radionuclides.

The present invention relates to a compound of formula (I) or a pharmaceutically acceptable salt thereof:

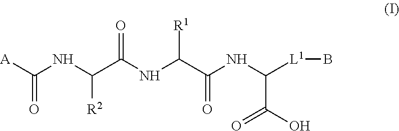

wherein
A is a group comprising a chelator group and a fluorescent dye,
R¹ is an —(C₀₋₆ alkylene)-(optionally substituted bicyclic aryl) or —(C₀₋₆ alkylene)-(optionally substituted bicyclic heteroaryl), wherein the aryl in said —(C₀₋₆ alkylene)-(optionally substituted bicyclic aryl) and the heteroaryl in said —(C₀₋₆ alkylene)-(optionally substituted bicyclic heteroaryl) are each optionally substituted with one or more groups R³;
R² is an —(C₀₋₆ alkylene)-(optionally substituted monocyclic aryl), —(C₀₋₆ alkylene)-(optionally substituted bicyclic aryl), —(C₀₋₆ alkylene)-(optionally substituted monocyclic heteroaryl), or —(C₀₋₆ alkylene)-((optionally substituted bicyclic heteroaryl), wherein the aryl in said —(C₀₋₆ alkylene)-(optionally substituted monocyclic aryl) and in said —(C₀₋₆ alkylene)-(optionally substituted bicyclic aryl) and the heteroaryl in said —(C₀₋₆ alkylene)-(optionally substituted monocyclic heteroaryl) and in said —(C₀₋₆ alkylene)-(optionally substituted bicyclic heteroaryl) are each optionally substituted with one or more groups R⁴;
each R³ is independently selected from halogen, C₁₋₆ alkyl, C₂₋₆ alkenyl, C₂₋₆ alkynyl, C₁₋₆ haloalkyl, —OR⁵, —SO₂R⁵, —NO₂, —CN, —C(O)R⁵, —C(O)OR⁵, —C(O)N(R⁵)₂, —NR⁵R⁵, —NCO, —NCS, —SR⁵, —N(R⁵)C(O)R⁵ and —N₂;
each R⁴ is independently selected from halogen, C₁₋₆ alkyl, C₂₋₆ alkenyl, C₂₋₆ alkynyl, C₁₋₆ haloalkyl, —OH, —OR⁵, —SO₂R⁵, —NO₂, —CN, —C(O)R⁵, —C(O)OR⁵, —C(O)N(R⁵)₂, NR⁵R⁵, —NCO, —NCS, —SR⁵, —NR⁵C(O)R⁵ and —N₂;
each R⁵ is independently selected from hydrogen, halogen, C₁₋₆ alkyl;
L¹ is a linear —(C₁₋₃₀ alkylene)- group, wherein one or more —CH₂— units in said alkylene are optionally replaced by a group independently selected from —O—, —NH—, —N(C₁₋₆ alkyl)-, —CHR⁶—, —C(R⁶)₂—, —CO—, —S—, —SO—, —SO₂— or —(C₃₋₆ cycloalkyl)-; wherein each R⁶ is independently selected from halogen, C₁₋₆ alkyl, C₂₋₆ alkenyl, C₂₋₆ alkynyl, C₁₋₆ haloalkyl, —OH, —O(C₁₋₆ alkyl), —C(O)C₁₋₆ alkyl, —CN, —NH₂, —NH(C₁₋₆ alkyl) and —N(C₁₋₆ alkyl)(C₁₋₆ alkyl);
and
B is selected from —EuK or —EuE;
wherein
—EuK is

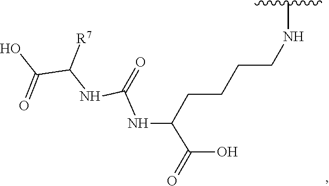

—EuE is

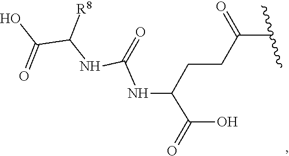

wherein R⁷ and R⁸ may be the same or different, wherein R⁷ and R⁸ are independently selected from —(C₁₋₆ alkylene)-C(O)OH, preferably wherein R⁷ and R⁸ are each —(C₂ alkylene)-C(O)OH;
wherein
the fluorescent dye is selected from Cy3, Cy3B, Cy3.5, Cy5, Cy5.5, Cy7, carbocyanine, indocarbocyanine, oxacarbocyanine, thiacarbocyanine, merocyanine, polymethine, coumarine, rhodamine, rhodamine B, xanthene, fluorescein, boron-dipyrromethane (BODIPY), VivoTag-680, Vivo Tag-S680, Vivo Tag-S750, AlexaFluor647, AlexaFluor660, AlexaFluor680, AlexaFluor700, AlexaFluor750, AlexaFluor790, Dy676, Dy677, Dy682, Dy752, Dy780, DyLight547, DyLight647, DyLight680, HiLyte Fluor 647, HiLyte Fluor 680, HiLyte Fluor 750, IRDye 800CW, IRDye 800RS, IRDye 700DX, ADS780WS, ADS830WS, ADS832WS and derivatives thereof.

In some embodiments, the invention relates to the compound of formula (I), wherein L¹ is selected from
(i) —(C₁₋₁₀ alkylene)-CONH—(C₁₋₁₀ alkylene)-CO—;
(ii) —(C₁₋₁₀ alkylene)-CONH—(C₁₋₁₀ alkylene)-NH—;
(iii) —(C₁₋₁₀ alkylene)-NHCO—(C₁₋₁₀ alkylene)-CO—; and
(iv) —(C₁₋₁₀ alkylene)-NHCO—(C₁₋₁₀ alkylene)-NH—.

In further embodiments, the invention relates to the compound of formula (I), wherein L¹ is selected from
(i) —(C₁₋₆ alkylene)-CONH—(C₁₋₆ alkylene)-CO—;
(ii) —(C₁₋₆ alkylene)-CONH—(C₁₋₆ alkylene)-NH—;
(iii) —(C₁₋₆ alkylene)-NHCO—(C₁₋₆ alkylene)-CO—; and
(iv) —(C₁₋₆ alkylene)-NHCO—(C₁₋₆ alkylene)-NH—.

In preferred embodiments, the invention relates to the compound of formula (I), wherein L¹ is —(C₁₋₄ alkylene)-NHCO—(C₁₋₆ alkylene)-CO—, more preferred is —(C₄ alkylene)-NHCO—(C₆ alkylene)-CO—.

The linker unit, such as L¹ has been described in the prior art as an important feature of PSMA targeting compounds. In particular, the linker has been described as necessary feature which facilitates an open conformation of the entrance lid of PSMA, thereby enabling accessibility to a remote arene binding site in PSMA, leading to an improvement in binding affinity (Zhang, A. X. et al., J. Am. Chem. Soc., 2010, 132, 12711-12716). It was further shown that the structural composition of the linker in combination with mas₃-based ^{99m}Tc-labeling also has a significant impact on pharmacokinetics and ultimately tumor-targeting. The prolonged circulation time observed for [^{99m}Tc]PSMA-I&S (Robu, S. et al., J. Nucl. Med., 2017, 58, 235-242) as compared to the corresponding PSMA-I&T-derived [⁶⁸Ga/¹⁷⁷Lu]DOTAGA-analog containing the same linker (Wirtz, M. et al., EJNMMI Res., 2018, 8, 84) is based on enhanced plasma protein binding (94 vs 81%, respectively) and leads to enhanced bioavailability at the tumor site. This results in higher absolute tumor uptake and thus improved imaging contrast (Liu, T., et al., Bioorg. Med. Chem. Lett., 2011, 21, 7013-7016), which are crucial for both high imaging quality and efficient lesion detection during radioguided surgery.

A fluorescent dye moiety, as used herein, may be understood in the broadest sense as any dye moiety enabling fluorescence detection. Preferably, such fluorescence detection is in a range of from 400 to 1000 nm, i.e. in the visible spectrum and in the Near Infrared (NIR) spectrum, in particular in a range of from 400 to 800 nm, i.e. in the visible spectrum. Preferably, the fluorescence signal emitted by the fluorescence dye moiety is well-distinguishable from the autofluorescence of the neoplasia and the surrounding tissue. Numerous fluorescent dye moieties are known in the art, and will be readily apparent to one of ordinary skill. Many fluorescent dyes are commercially available with activated groups used to react with protein sidechains or other compounds such as precursors to the compound of the present invention. Preferably, the fluorescence dye moiety in the context of the present invention is a small-molecule dye, i.e., a fluorescence dye moiety having a molecular weight (MW) of not more than 1000 Da, preferably not more than 750 Da, in particular not more than 500 Da. The skilled person is aware of the hereinabove described fluorescent dyes, which are commonly used or incorporated into compounds for medical imaging. Appropriate derivatives of the hereinabove described dyes are readily recognized by a skilled person, said dyes may be identified and purchased from commercial libraries comprising many of the hereinabove described dyes and their derivatives.

In preferred embodiments a linker is also a part of the group A in the compound of formula (I), accordingly the compound of formula (I) preferably has a general formula:

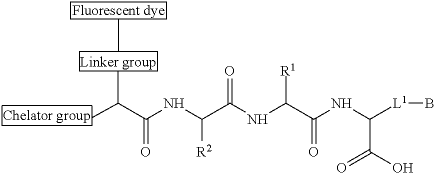

Linker groups are groups which separate two parts of a molecule. In the present invention, the linker group forms covalent bonds with both the fluorescent dye and the part of the structure of the compound of formula (I) which is different from A, The linker group may, in principle, be any chemical group which is capable of forming bonds with both the fluorescent dye and the part of the structure of the compound of formula (I) which is different from A. Preferably, the linker group contains only atoms selected from H, B, C, N, O, F, Si, P, S, Cl, Br and I. Examples of linker groups which can be used in the present invention contain one or more groups selected from —O—, —NH—, —N(C₁₋₆ alkyl)-, —CHR⁶—, —C(R⁶)₂—, —CO—, —S—, —SO—, —SO₂— or a —(C₁₋₁₅ alkylene)- group, wherein one or more —CH₂— units in said alkylene are optionally replaced by a group independently selected from —O—, —NH—, —N(C₁₋₆ alkyl)-, —CHR⁶—, —C(R⁶)₂—, —CO—, —S—, —SO—, —SO₂— or —(C₃₋₆ cycloalkylene)-; wherein each R⁶ is independently selected from halogen, C₁₋₆ alkyl, C₂₋₆ alkenyl, C₂₋₆ alkynyl, C₁₋₆ haloalkyl, —OH, —O(C₁₋₆ alkyl), —C(O)C₁₋₆ alkyl, —CN, —NH₂, —NH(C₁₋₆ alkyl) and —N(C₁₋₆ alkyl)(C₁₋₆ alkyl). Preferably the linker group which forms covalent bonds with both the fluorescent dye and the part of the structure of the compound of formula (I) which is different from A is —(C₁₋₄ alkylene)-NHCO—(C₁₋₅ alkylene)-, more preferably the linker is —(C₄ alkylene)-NHCO—(C₅ alkylene)-.

In preferred embodiments, the invention relates to a compound of formula (I) wherein the fluorescent dye is Cy5, Cy 5.5, Cy7 or a derivative thereof. The skilled person is aware of derivatives of the cyanine dyes, Cy5, Cy5.5 or Cy7. Exemplary and preferred examples include non-sulfonated and sulfonated derivatives such as

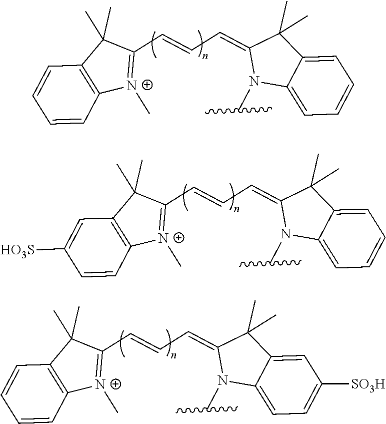

wherein n is 2 or 3. In preferred embodiments the cyanine dye is a di-sulfonated derivative of Cy5, having the formula

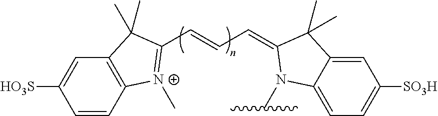

wherein n is 2 or 3, preferably wherein n is 2.

As mentioned hereinabove, the compound of formula (I) comprises A, which is a group comprising a chelator group and a fluorescent dye. The chelator group is not particularly limited and thus may be any chelator group known to a person skilled in the art, in particular the chelator group may be a chelator known to a person skilled art that is capable of forming a complex (by chelating) with a radionuclide used in imaging, diagnosis and/or therapy.

Moreover, the group A may comprise a linker group and a chelator group, wherein the compound of formula (I) has the following general formula:

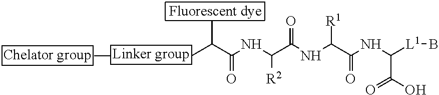

The linker group as depicted hereinabove, forms covalent bonds with both the chelator group and the part of the structure of the compound of formula (I) which is different from A, The linker group may, in principle, be any chemical group which is capable of forming bonds with both the chelator group and the part of the structure of the compound of formula (I) which is different from A. Preferably, the linker group contains only atoms selected from H, B, C, N, O, F, Si, P, S, Cl, Br and I, which are preferably different from the radionuclides defined herein. Examples of linker groups which can be used in the present invention contain one or more groups selected from —O—, —NH—, —N(C₁₋₆ alkyl)-, —CHR⁶—, —C(R⁶)₂—, —CO—, —S—, —SO—, —SO₂— or a —(C₁₋₁₀ alkylene)- group, wherein one or more —CH₂— units in said alkylene are optionally replaced by a group independently selected from —O—, —NH—, —N(C₁₋₆ alkyl)-, —CHR⁶—, —C(R⁶)₂—, —CO—, —S—, —SO—, —SO₂— or —(C₃₋₆ cycloalkylene)-; wherein each R⁶ is independently selected from halogen, C₁₋₆ alkyl, C₂₋₆ alkenyl, C₂₋₆ alkynyl, C₁₋₆ haloalkyl, —OH, —O(C₁₋₆ alkyl), —C(O)C₁₋₆ alkyl, —CN, —NH₂, —NH(C₁₋₆ alkyl) and —N(C₁₋₆ alkyl)(C₁₋₆ alkyl). Preferably the linker group is an amino group. The amino group preferably forms an amide group with a carboxylic acid of the chelator group.

Furthermore, as will be apparent to a skilled person, two adjacent groups in the linker group which forms covalent bonds with the chelator group or fluorescent dye and the part of the molecule which is different from A, should be chosen so as to avoid a direct bond between two groups which would result in a partial structure which is not stable, in particular, in an aqueous medium at 25° C. and a pressure of 1 atm. From this standpoint, combinations such as —N(R⁴)—N(R⁴)—, —C(O)—C(O)—, —O—O—, —S—S—, —S(O)—S(O)—, —S(O)₂—S(O)₂—, —N(R⁴)—O—, —O—N(R⁴)—, —N(R⁴)—S—, —S—N(R⁴)—, —N(R⁴)—S(O)—, —S(O)—N(R⁴)—, —C(O)—S—, —S—C(O)—, —C(O)—S(O)—, —S(O)—C(O)—, —C(O)—S(O)₂—, —S(O)₂—C(O)—, —S—O—, —O—S—, —S(O)—O—, —O—S(O)—, —S(O)—S—, —S—S(O)—, —S(O)₂—S—, —S—S(O)₂—, —S(O)₂—S(O)— and —S(O)—S(O)₂— are preferably excluded.

In preferred embodiments, the invention relates to the compound of formula (I), wherein the chelator group is selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]-hexadecan (D02A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphate) (DPDP), diethylenetriaminepentaacetic acid (DTPA), ethylenediamine-N,N′-tetraacetic acid (EDTA), ethylene glycol-0,0-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridin-bis(methyl-enamine)tetraacetic acid (TMT), 1,4,7,10-tetraazacyclo-tridecan-N,N′,N″,N′″-tetraacetic acid (TRITA), triethylenetetraaminehexaacetic acid (TTHA), N,N′-bis[(6-carboxy-2-pyridyl)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl}heptanedioic acid bis-[(3-hydroxy-1,6-dimethyl-4-oxo-1,4-dihydro-pyridin-2-ylmethyl)-amide](THP), mercaptoacetyl triserine (mas₃), mercaptoacetyl triglycine (mag₃) and derivatives thereof.

These chelator groups may be attached to the linker group or the part of the structure of the compound of formula (I) which is different from A in any manner. For example, the chelator group may be attached to a linker group or the part of the structure of the compound of formula (I) which is different from A via a nitrogen atom within a heterocyclic ring or chain of the chelator group or via a carboxylic acid in one of the side chains of the chelator group.

In preferred embodiments, the invention relates to a compound of formula (I), wherein
B is EuK having the configuration

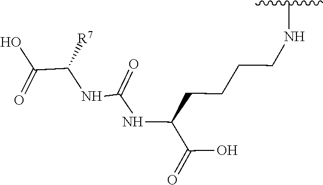

or EuE having the configuration

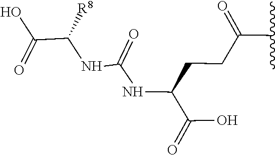

wherein R⁷ and R⁸ may be the same or different, wherein R⁷ and R⁸ are each independently selected from —(C₁₋₆ alkylene)-C(O)OH, preferably wherein R⁷ and R⁸ are each —(C₂ alkylene)-C(O)OH.

The inventors of the present invention found that it is particularly advantageous to have the group B, which represents a PSMA targeting moiety, as EuK or EuE, in particular where EuK or EuE is in the hereinabove depicted configuration. The advantageous effect of EuK or EuE is an increased affinity and specificity to the PSMA binding site. The targeting moiety utilizes predominantly the aspartate (in the S1, also known as nonprime position) and glutamate (ST, also known as S1′ or prime position) binding sites, while urea can coordinate to Zn²⁺ comprised in the ST region.

In particularly preferred embodiments, the invention relates to compounds of formula (I), wherein the chelator group is -mas₃ or a thioether or thioester derivative thereof, -mag₃ or a thioether or thioester derivative thereof, -DOTAGA or -DOTA.

The chelator group -mas₃ refers to mercaptoacetyl triserine, which comprises three serine residues and has the general formula

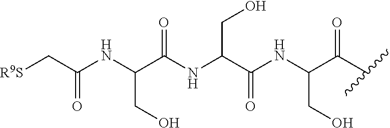

wherein R⁹ is hydrogen or a thiol protecting group, preferably a thioether or thioester protecting group, such as an acetyl protecting group, a benzyl protecting group, a benzoyl protecting group or any thiol protecting group known to one skilled in the art (See, Greene and Wuts, Protecting Groups in Organic Synthesis, Third Edition, John Wiley & Sons (1999)). The term “thiol protecting group”, as used herein, refers to a moiety that temporarily blocks a thiol reactive site in a compound. Generally, this is done so that a chemical reaction can be carried out at another reactive site in a multifunctional compound or to otherwise stabilize the thiol. In one embodiment, the thiol protecting group is selectively removable by a chemical reaction.

The chelator group -mas₃ may comprise each serine residue as the D- or L-isomer, accordingly -mas₃ may comprise all D- or all L-serine residues or a mixture of said residues. D- and L-forms of serine have the following configuration

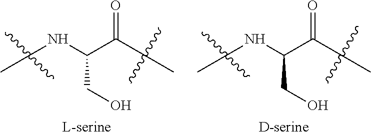 [SMILES: *N[C@@H](CO)C(*)=O ; *N[C@H](CO)C(*)=O]

In preferred embodiments, the invention relates to a compound of formula (I) having -mas₃ as the chelator group, wherein -mas₃ comprises all D- or all L-serine residues, most preferred is -mas₃ comprising all D-serine residues. Accordingly, -mas₃ or a thioether or thioester derivative of -mas₃ is preferably selected from

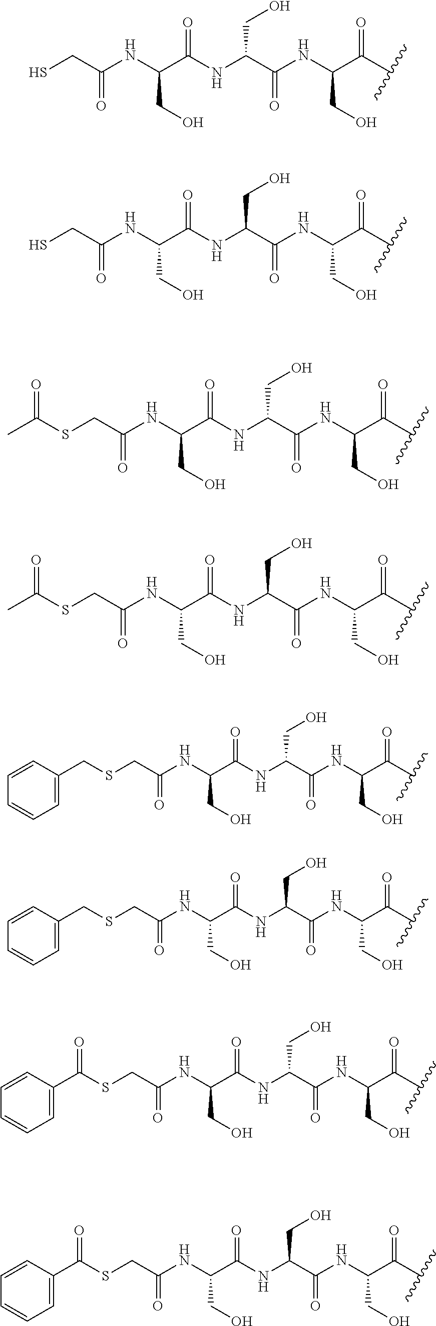

In further embodiments, the invention relates to a compound of formula (I) wherein the chelator group is -mag₃, or mercaptoacetyl triglycine, having the general formula

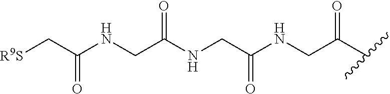

wherein R⁹ is hydrogen or a thiol protecting group, preferably a thioether or thioester protecting group, such as an acetyl protecting group, a benzyl protecting group, a benzoyl protecting group or any thiol protecting group known to one skilled in the art (See, Greene and Wuts, Protecting Groups in Organic Synthesis, Third Edition, John Wiley & Sons (1999)). The term “thiol protecting group”, as used herein, refers to a moiety that temporarily blocks a thiol reactive site in a compound. Generally, this is done so that a chemical reaction can be carried out at another reactive site in a multifunctional compound or to otherwise stabilize the thiol. In one embodiment, the thiol protecting group is selectively removable by a chemical reaction. Accordingly, in preferred embodiments -mag₃ or a thioether or thioester derivative of -mag₃ selected from

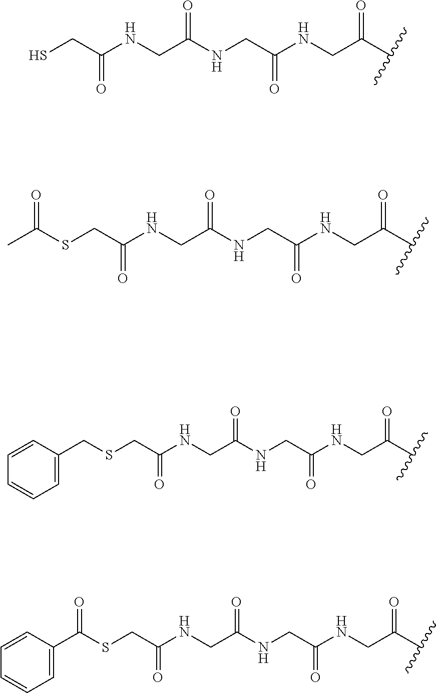 [SMILES: *C(=O)CNC(=O)CNC(=O)CNC(=O)CS ; *C(=O)CNC(=O)CNC(=O)CNC(=O)CSC(C)=O ; *C(=O)CNC(=O)CNC(=O)CNC(=O)CSCc1ccccc1 ; *C(=O)CNC(=O)CNC(=O)CNC(=O)CSC(=O)c1ccccc1]

In some embodiments, the invention relates to compounds of formula (I), wherein the chelator group is -DOTAGA, having the following formula

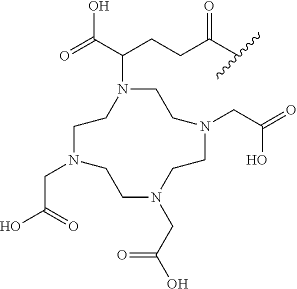 [SMILES: *C(=O)CCC(C(=O)O)N1CCN(CC(=O)O)CCN(CC(=O)O)CCN(CC(=O)O)CC1]

In some embodiments, the invention relates to compounds of formula (I), wherein the chelator group is -DOTA, having the following formula

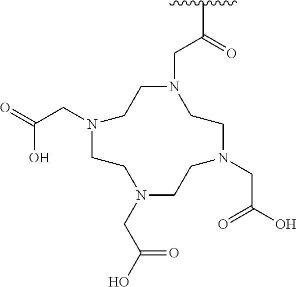 [SMILES: *C(=O)CN1CCN(CC(=O)O)CCN(CC(=O)O)CCN(CC(=O)O)CC1]

In preferred embodiments, the invention relates to the compound of formula (I), wherein R¹ is a —(C₁ alkylene)-(optionally substituted bicyclic 10-membered aryl) and R² is a —(C₁ alkylene)-(optionally substituted monocyclic 6-membered aryl), wherein the bicyclic 10-membered aryl in said —(C₁ alkylene)-(optionally substituted bicyclic 10-membered aryl) is each optionally substituted with one or more groups R³, and the monocyclic 6-membered aryl in said —(C₁ alkylene)-(optionally substituted monocyclic 6-membered aryl) is substituted with an R⁴ group —OH. In preferred embodiments the compound of formula (I) has the following structure (compound of formula (I′)):

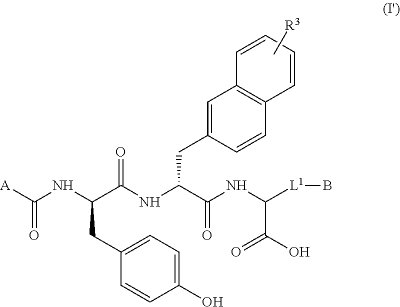

The inventors of the present invention found that the compound of formula (I) having the structure and configuration depicted hereinabove is particularly advantageous in targeting PSMA. This is due to the fact that the substituted bicyclic 10-membered aryl is able to interact with a remote arene binding site in PSMA, which results in an increase in the inhibitor affinity. The PSMA-binding of the targeting molecule, i.e. the compound of formula (I), via the inhibitor component is strengthened by the additional favourable interaction of the linker with the remote binding pocket (reduced k_{off}). Accordingly, the choice of this particular linker, i.e., R¹ and R² as depicted in the compound of formula (I) and the specific R¹ and R² as depicted in the compound of formula (I′) not only influences the hydrophobicity or lipophilicity of the compounds described, but has the unexpected effect of improving the compound's capacity to target a remote arene binding site in the PSMA molecule and thus to improve ligand affinity, in particular with respect to the R¹. The group R¹ has thus been selected for efficient interaction with the PSMA binding pocket, via, inter alia, pi-pi interactions and lipophilic interactions.

In preferred embodiments, the compound of formula (I′) is a compound wherein:
A is a group comprising a chelator group and a fluorescent dye, preferably wherein the chelator group is selected from is -mas₃ or a thioether or thioester derivative thereof, -mag₃ or a thioether or thioester derivative thereof, -DOTAGA or -DOTA, more preferably wherein chelator group is selected from is -mas₃ or a thioether or thioester derivative thereof, -mag₃ or a thioether or thioester derivative thereof. Preferably, the fluorescent dye is selected from is Cy5, Cy 5.5, Cy7 or a derivative thereof, more preferably wherein the wherein the fluorescent dye is a derivative of Cy5, Cy7 or derivatives thereof, preferably selected from

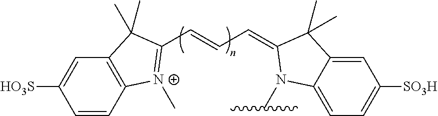

wherein
n is 2 or 3, preferably n is 2.

In further preferred embodiments of the compound of formula (I′), L¹ is a linear —(C₁₋₃₀ alkylene)- group as described hereinabove for the compound of formula (I), preferably L¹ is selected from
(i) —(C₁₋₁₀ alkylene)-CONH—(C₁₋₁₀ alkylene)-CO—;
(ii) —(C₁₋₁₀ alkylene)-CONH—(C₁₋₁₀ alkylene)-NH—;
(iii) —(C₁₋₁₀ alkylene)-NHCO—(C₁₋₁₀ alkylene)-CO—; and
(iv) —(C₁₋₁₀ alkylene)-NHCO—(C₁₋₁₀ alkylene)-NH—.

More preferably wherein L¹ is selected from
(i) —(C₁₋₆ alkylene)-CONH—(C₁₋₆ alkylene)-CO—;
(ii) —(C₁₋₆ alkylene)-CONH—(C₁₋₆ alkylene)-NH—;
(iii) —(C₁₋₆ alkylene)-NHCO—(C₁₋₆ alkylene)-CO—; and
(iv) —(C₁₋₆ alkylene)-NHCO—(C₁₋₆ alkylene)-NH—.

The group B in the compound of formula (I′) is selected from —EuK or —EuE as described hereinabove for the compound of formula (I).

The inventors of the present invention have surprisingly found that the combination of the above-described features for the compound of formula (I′) displays particularly improved pharmacokinetic properties in terms of, inter alia, circulation time and tumour accumulation as described in the appended examples.

In further preferred embodiments, the invention relates to the compound of formula (I), wherein the compound is selected from

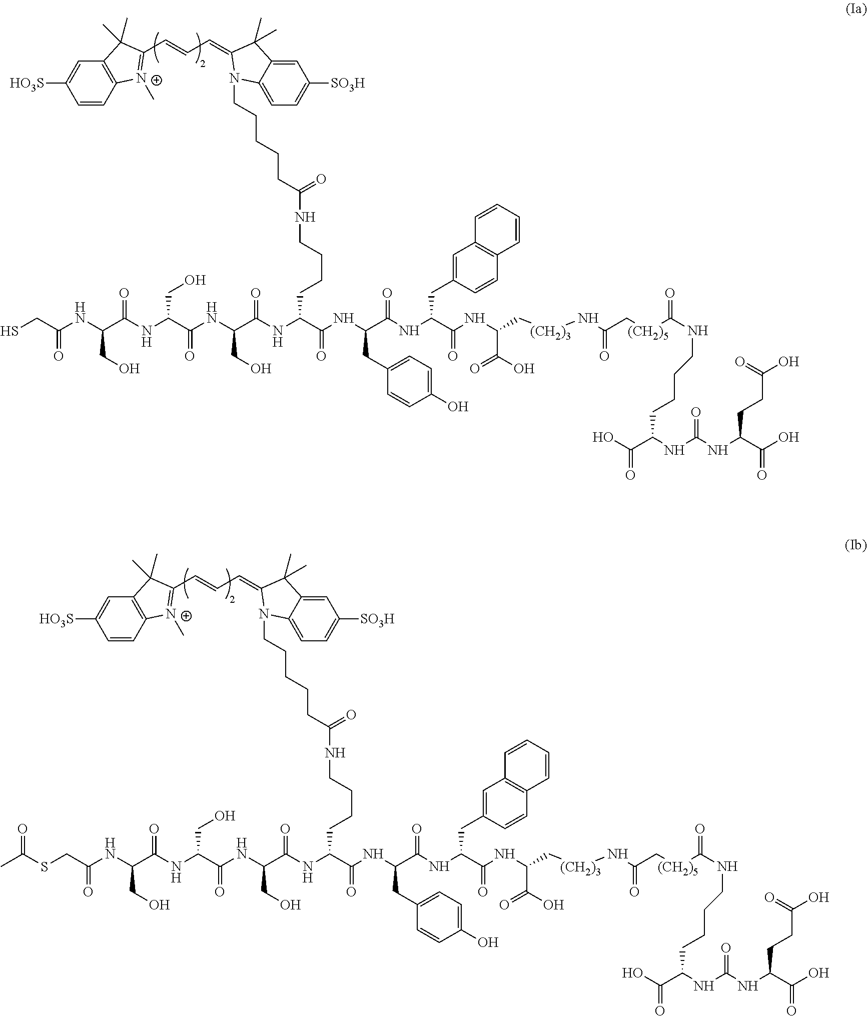

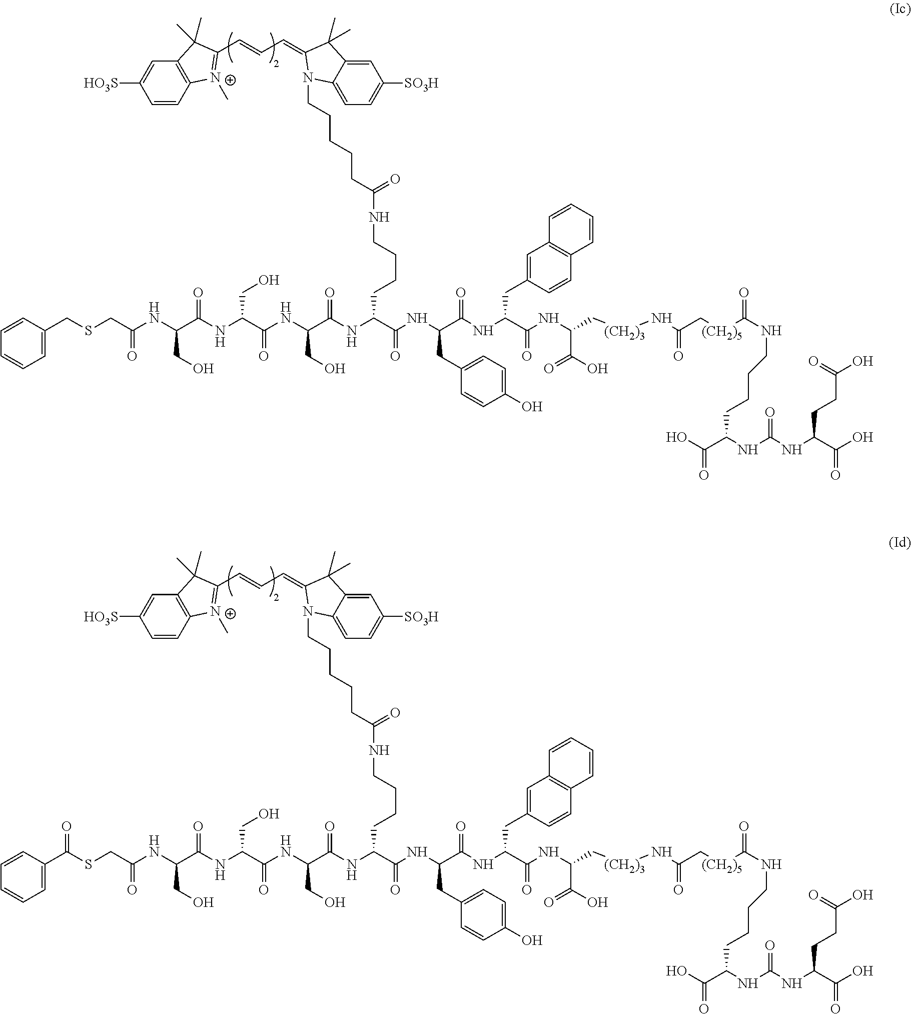

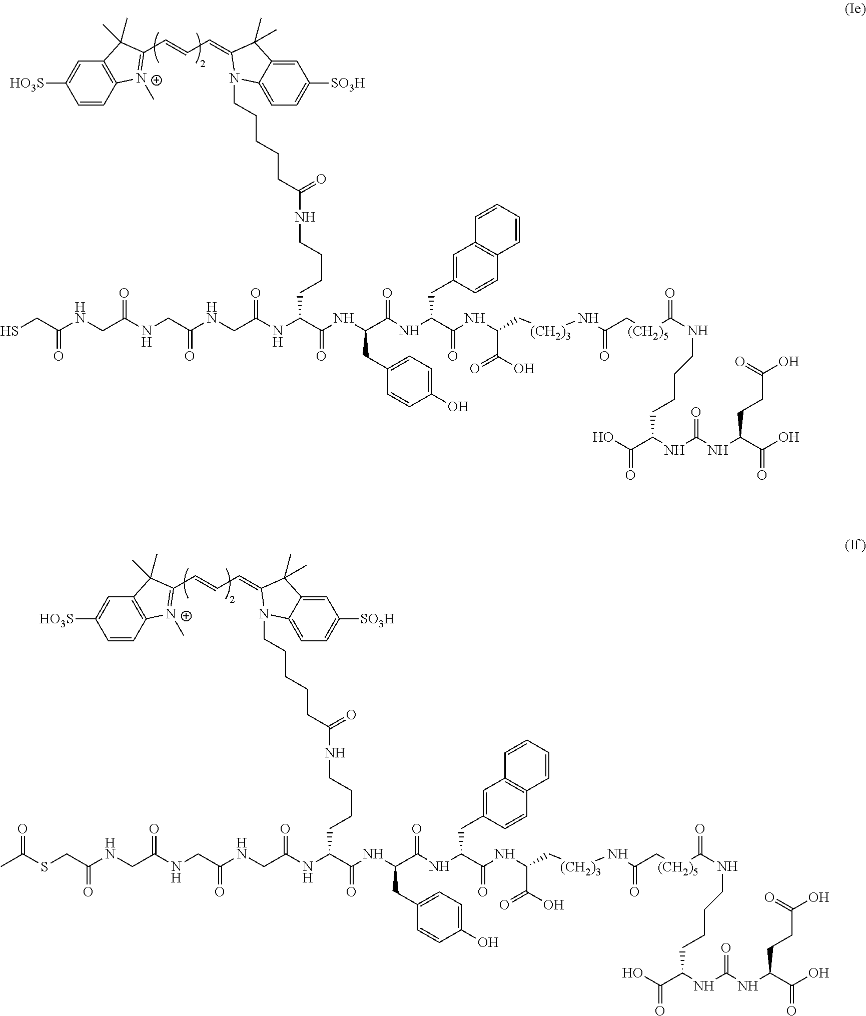

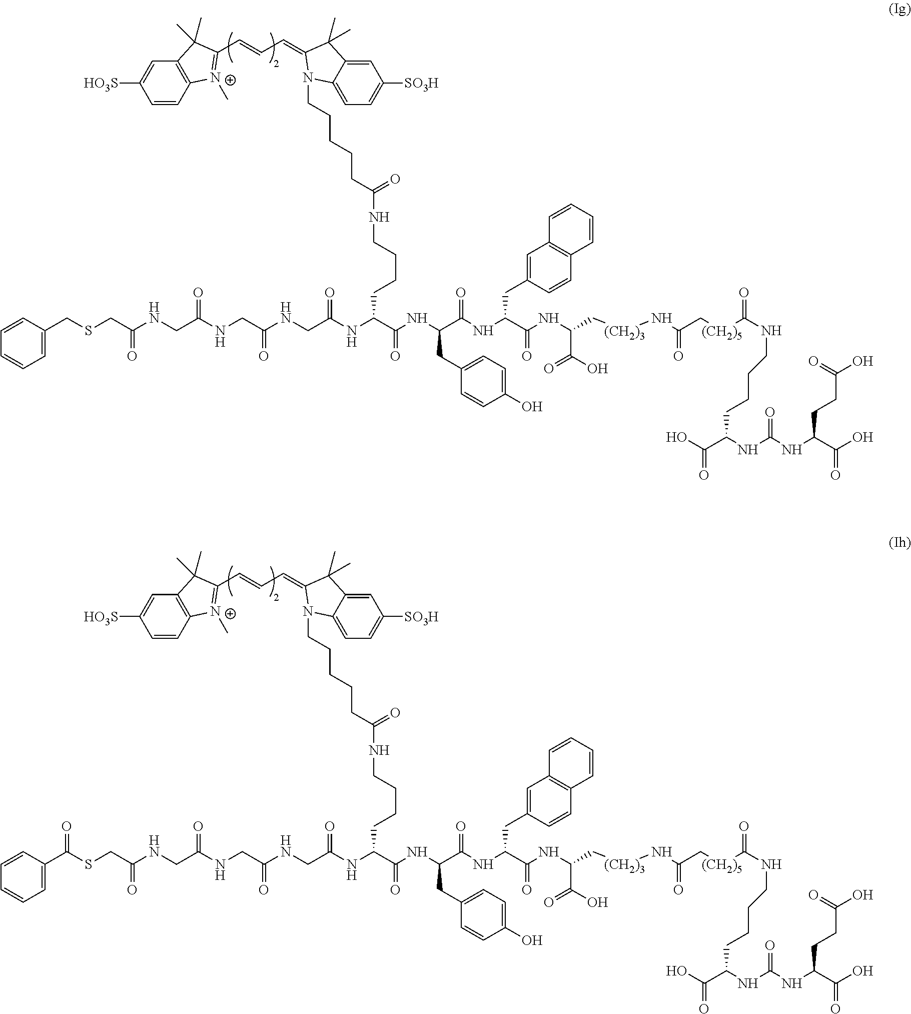

Most preferred is a compound of formula (I) having the structure

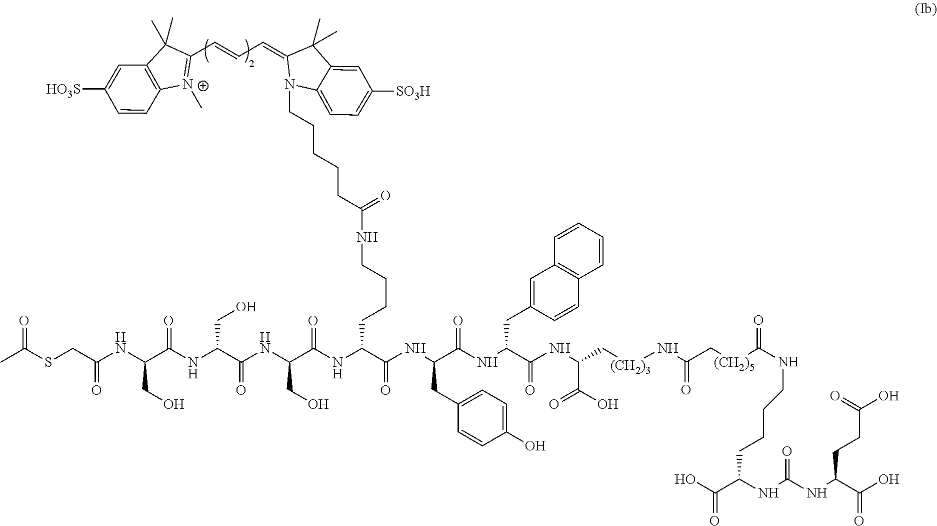

The inventors of the present invention surprisingly found that the compounds of formula (I′) and (Ia)-(Ih) having the composition as described hereinabove, in particular the selection of the chelator group, the fluorescent dye, the linker comprising the R¹ and R² groups as described and the group B displays improved circulation time and high tumor accumulation. As demonstrated in the appended examples, this combination of features displays outstanding tumor accumulation compared to previously described compounds, where the linker has high PSMA targeting affinity, the fluorescent dye has negligible influence on said affinity and the chelator group displays slow clearance kinetics. This combination of features results in 50% enhanced tumor accumulation for compound (Ib) as described in the appended examples, which provides an optimal signal intensity in tumor lesions during radio- and fluorescence guided surgery, thus further improving the sensitivity of the method.

In further preferred embodiments, the invention relates to a composition comprising the compound of formula (I) and a radionuclide, preferably wherein the radionuclide is selected from ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁷Ga, ⁶⁸Ga, ⁸⁹Zr, ⁸⁹Y, ⁹⁰Y, ^{94m}Tc, ^{99m}Tc, ⁹⁷Ru, ¹⁰⁵Rh, ¹⁰⁹Pd, ¹¹¹Ag, ¹¹⁰In, ¹¹¹In, ¹¹³In, ¹¹⁴In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁷Lu, ¹⁸⁶Re, ¹⁸⁸Re, ¹⁹¹Pt, ¹⁹⁷Hg, ¹⁹⁸Au, ¹⁹⁹Au, ²⁰³Pb, ²¹²Pb, ²¹¹At, ²¹²Bi, ²¹³Bi, ²²³Ra, ²²⁵Ac, and ²²⁷Th, and a cationic molecule comprising ¹⁸F, such as ¹⁸F—[AlF]²⁺. As is understood by the person skilled in the art, the hereinabove described radionuclides may be used for therapeutic and/or diagnostic and/or imaging. Preferred radionuclides disclosed herein for imaging and/or diagnostic use are ^{99m}Tc, ⁶⁴Cu, ⁶⁸Ga, ¹¹¹In and ¹⁸F, such as ¹⁸F—[AlF]²⁺. Therefore, in preferred embodiments the invention relates to a composition comprising the compound of formula (I) and a radionuclide selected from ^{99m}Tc, ⁶⁴Cu, ⁶⁸Ga, ¹¹¹In and ¹⁸F, such as ¹⁸F—[AlF]²⁺ for use in the imaging and/or diagnosis of a disease, in particular a solid cancer. Preferred radionuclides disclosed herein for imaging and/or diagnosis and/or therapeutic use are ¹⁷⁷Lu, ⁹⁰Y, ⁶⁷Cu, ¹⁵³Sm, ¹⁶¹Tb, ¹⁸⁸Re, ²¹²Pb and ²²⁵Ac. Accordingly, in preferred embodiments, the invention relates to a composition comprising the compound of formula (I) and a radionuclide selected from ¹⁷⁷Lu, ⁹⁰Y ⁶⁷Cu, ¹⁵³Sm, ¹⁶¹Tb, ¹⁸⁸Re, ²¹²Pb and ²²⁵Ac for use in the imaging and/or diagnosis and/or treatment of a disease, in particular a solid cancer.

The inventors of the present invention found that a composition comprising a compound of formula (I) and a radionuclide are particularly suitable as tracers. In particular, compositions of the invention combine a probe (e.g. a PSMA probe) with a fluorophore and moiety, such as a chelator, that may be radiolabeled, e.g. by chelating a radionuclide (i.e. a radioactive isotope). These compositions, when they comprise a radionuclide, may be considered as a hybrid tracer, as the probe portion is conjugated to two labels that can serve a complementary purpose: a fluorophore and a radiolabel. The efficacy of such a tracer can be optimized by fine-tuning individual portions of the compounds. At the same time, these findings also suggest that this design concept remains valid when e.g. the targeting moiety, dye, or chelator is varied. For example, the dye portion may comprise a Cy5, Cy7 or a derivative thereof, and the chelator portion may comprise a chelating moiety as disclosed herein, such as mas₃ or DOTAGA. The choice of the dye influences the wavelength used for surgical guidance (e.g. far red for Cy5 or near-infrared for Cy7). Appropriate choice of the chelator portion aims to accommodate a specific radionuclide or accommodate various different radionuclides. For example, a chelator that may be used with an alpha emitter (e.g. ²²⁵Ac, ²²⁴Ra, ²¹³Bi), may be suitable for use with therapeutic isotopes; while a chelator that may be used with a gamma emitter (e.g. ^{99m}Tc, ¹¹¹In), may be suitable for use with radiolabel imaging isotopes. Most chelators are able to coordinate different radionuclides. For example, DOTAGA may chelate ¹¹¹In, ⁶⁸Ga, ¹⁷⁷Lu, etc.; and mas₃ may chelate ^{99m}Tc, ¹⁸⁸Re, etc. The skilled person is able to determine which chelator is suited to which radionuclide (see e.g. Price, E. W. and Orvig, C., Chem. Soc. Rev., 2014, 43, 260-290; Sarko, D. et al., Current Med. Chem., 2012, 19, 2667-2688; Abiraj, K. et al., Chem. Eur. J., 2010, 16, 2115-2124).

The term “probe” or “targeting moiety” is to be understood as referring to a moiety that targets PSMA via an affinity type interaction. Any probe known to the skilled person may be a part of B in the compound of formula (I) as long it targets PSMA; many examples including monoclonal antibodies and low-molecular weight moieties have been described in the art (see e.g. Fakiri, M. E. et al., Cancers, 2021, 13, 3967). The term “low-molecular weight moiety”, as used herein, refers to moieties with a molecular weight less than 800 Dalton. Exemplary probes include moieties of formula EuX, namely glutamate linked to another amino acid or another low-molecular weight moiety via a bridging urea, for example EuK (glutamate-urea-lysine), EuE (glutamate-urea-glutamate), EuFA, which has the formula

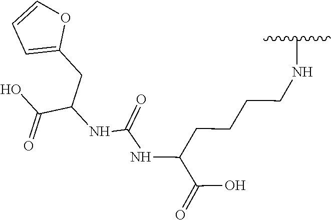 [SMILES: *NCCCCC(NC(=O)NC(Cc1ccco1)C(=O)O)C(=O)O]

or EuPG which has the formula

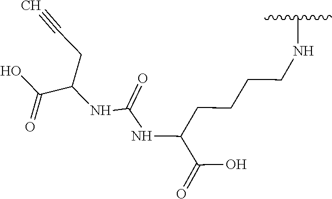 [SMILES: *NCCCCC(NC(=O)NC(CC#C)C(=O)O)C(=O)O]

In preferred embodiments, the compound of formula (I) contains a probe in portion B which is EuK or EuE, wherein EuK is the most preferred.

The term “tracer”, as used herein, refers to a molecule comprising a targeting moiety/probe, such as EuE and an imaging label. An imaging label may be any molecule or compound which may be detected by imaging with a camera or microscope, e.g. a gamma camera or spectrometer. A tracer may comprise two imaging labels, e.g. as a hybrid tracer with two different types of imaging label. In a hybrid tracer, a probe portion may be conjugated to two labels that can serve a complementary purpose, for example a combination of a fluorophore label and a radiolabel. The term “radiolabel”, as used herein, refers to the portion of the molecule comprising a chelator and a radionuclide.

In preferred embodiments, the invention relates to the composition of the compound of formula (I) and a radionuclide, wherein the radionuclide is ^{99m}Tc.

The inventors of the present invention found that it is particularly advantageous to use [^{99m}Tc]mas₃ as the radiolabel due to the improved tumor targeting as a result of a longer circulation time. This also resulted in a high tumor/background ratio at late time points (e.g. 4 to 21 h p.i.). ^{99m}Tc is also a low cost readily available radionuclide, therefore easily accessible in clinical settings. Finally, ^{99m}Tc has excellent nuclear properties as it emits readily detectable gamma rays with a photon energy of 140 kV and its half-life for gamma emission is 6.0058 h (meaning 93.7% of it decays to ⁹⁹Tc in 24 h). The physical half-life of the isotope and its biological half-life of 24 h, mean that it is highly useful for short procedures while keeping the total patient radiation exposure low.

In some embodiments, the invention relates to a composition, wherein the compound of formula (I) comprises a chelator group having at least 2, preferably 3, more preferably 4 functional groups for bonding to the radionuclide ^{99m}Tc, wherein said functional group comprises an atom having 1 to 3 free electron lone pair(s), wherein said atom is O, P, N or S; and wherein the chelator group is preferably bidentate, tridentate or tetradentate; and/or wherein the chelator group is cyclic or acyclic.

Exemplary chelator groups include, but are not limited to, diethylenetriaminepentaacetic acid (DTPA), 6-hydrazinyl-N-methylpyridine-3-carboxamide (HYNIC), ethylenediamine diacetic acid (EDDA), N-(2-hydroxy-1,1-bis(hydroxymethyl)ethyl)glycine (tricine), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA), mercaptoacetyl triserine (mas₃), mercaptoacetyl triglycine (mag₃) and derivatives thereof, and compounds having the following structures:

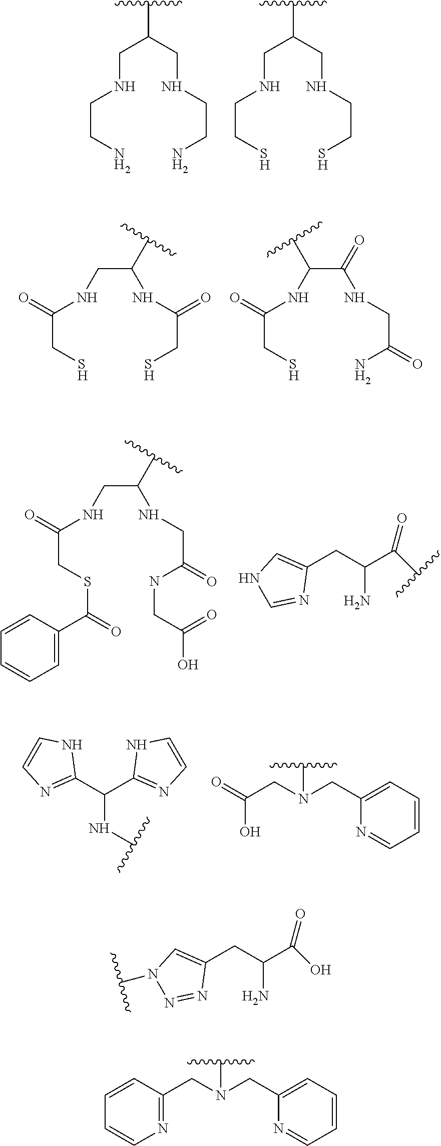

and derivatives thereof. In preferred embodiments the chelator group is mercaptoacetyl triserine (mas₃), mercaptoacetyl triglycine (mag₃) and derivatives thereof, even more preferably the chelator group is mas₃. It is to be understood that “ [symbol] ” represents the point of attachment to the remainder of the compound.

As can be seen from the exemplary structures and compounds hereinabove, the chelators may be cyclic or acyclic, one example of a cyclic chelator group is e.g., DOTA, an example of an acyclic chelator group is e.g., mas₃.

In some embodiments, the invention relates to a pharmaceutical composition comprising the compound of formula (I) or the composition comprising the compound of formula (I) and a radionuclide and a pharmaceutically acceptable excipient.

In further embodiments, the invention relates to a compound of formula (I), the composition or the pharmaceutical composition for use in diagnostics, preferably for use in imaging, preferably for use in imaging a solid cancer.

Moreover, in some embodiments, the invention relates to the compound of formula (I), the composition or the pharmaceutical composition for use in diagnostics, wherein the solid cancer is selected from lung cancer, gastrointestinal cancer, colorectal cancer, colon cancer, anal cancer, liver cancer, pancreatic cancer, stomach cancer, genitourinary cancer, bladder cancer, biliary tract cancer, hepatobiliary cancer, testicular cancer, cervical cancer, ovarian cancer, uterine cancer, endometrial cancer, vaginal cancer, vulvar cancer, malignant mesothelioma, oesophageal cancer, laryngeal cancer, prostate cancer, breast cancer, brain cancer, neuroblastoma, Ewing's sarcoma, osteogenic sarcoma, kidney cancer, epidermoid cancer, skin cancer, melanoma, head and/or neck cancer, mouth cancer, thymoma, Merkel-cell cancer, and neuroendocrine cancer, preferably wherein the solid cancer is prostate cancer.

In preferred embodiments, the invention relates to the compound of formula (I), the composition or the pharmaceutical composition for use in diagnostics, wherein the solid cancer may be a cancer comprising cells that express PSMA. For example, the cancer may be prostate cancer, renal cancer, breast cancer, gliomas, colorectal adenocarcinoma, transitional cell carcinoma, pancreatic ductal adenocarcinoma, or gastric adenocarcinoma; e.g. the cancer may be prostate cancer.

In some embodiments, the invention relates to the compound of formula (I), the composition or the pharmaceutical composition for use in diagnostics, wherein the use comprises administering the compound of formula (I), the composition or the pharmaceutical composition to a subject and thereafter imaging the tumour.

The term “imaging” preferably refers to the visual representation of a sample by detecting radiation. The radiation may be a product of radioactive decay of a radionuclide. The radiation may be a product of fluorescence. The visual representation may be provided by electronic processing of the detected radiation, for example by performing positron emission spectroscopy (PET), single photon emission computed tomography (SPECT), scintigraphy, (optionally intraoperative) gamma-tracing/imaging, (optionally intraoperative) beta-tracing. The visual representation may be provided by visual detection, for example by observing samples that fluoresce at visible wavelengths (e.g. from about 390 to 700 nm) on exposure to light of a shorter wavelength than that emitted by the fluorophore. The visual representation may be provided by fluorescence spectroscopy, wherein, typically, the fluorophore is exposed to ultraviolet light resulting in the emission of visible light.

The imaging may comprise imaging of radioactive decay and fluorescence imaging. The imaging may comprise imaging of radioactive decay prior to fluorescence imaging. For example, the imaging of radioactive decay may be performed before surgery and the fluorescence imaging may be performed during surgery.

The method of imaging may further comprise administering to said subject at least one compound that blocks or reduces the uptake of the compound of the invention in an organ (or in multiple organs). This may reduce the background signal of the compound of the invention in the relevant organ(s). For example, the at least one compound may block or reduce the uptake of the compound of the invention in kidneys and/or salivary glands. Examples of such compounds include mannitol, 2-(phosphonomethyl)pentanedioic acid, monosodium glutamate, succinylated gelatin and albumin fragments. The at least one blocking compound may be co-administered with the compound of the invention (e.g. as a single dosage form), or the at least one blocking compound may be administered separately to the compound of the invention.

In preferred embodiments, the invention relates to the compound of formula (I), the composition or the pharmaceutical composition for use in diagnostics, wherein the imaging comprises positron emission spectroscopy (PET), single photon emission computed tomography (SPECT), scintigraphy, (intraoperative)gamma-tracing/imaging, or (intraoperative)beta-tracing; and wherein the imaging comprises fluorescence imaging, optionally wherein the imaging comprises fluorescence spectroscopy.

In some embodiments, the invention relates to an in vitro method of imaging tissues expressing or over-expressing prostrate specific membrane antigen (PSMA), the method comprising contacting said tissue with the compound of formula (I), the composition or the pharmaceutical composition of the present invention.

The term “contacting”, as used herein, refers to the exposure of the tissue or cells thereof, to the compound or composition of the present invention. Contacting herein preferably refers to targeting said tissue or cell thereof with the compound or composition of the present invention, wherein PSMA is targeted by the probe/targeting moiety part of the compound of formula (I) which targets PSMA via an affinity type interaction.

In further embodiments, the invention relates to the use of the compound of formula (I), the composition or the pharmaceutical composition of the present invention as an imaging agent.

In preferred embodiments, the invention relates to the use of the compound of formula (I), the composition or the pharmaceutical composition as an imaging agent for imaging a solid cancer, in particular wherein the solid cancer may be a cancer comprising cells that express PSMA. For example, the cancer may be prostate cancer, renal cancer, breast cancer, gliomas, colorectal adenocarcinoma, transitional cell carcinoma, pancreatic ductal adenocarcinoma, or gastric adenocarcinoma; preferably the cancer is prostate cancer.

In some embodiments, the invention provides a method for imaging a tumor, comprising administering to a subject a compound of formula (I), the composition or the pharmaceutical composition of the present invention, and after a predetermined time imaging the tumor. The predetermined time is determined based on numerous factors, including, inter alia, the mode of administration of the compound, composition or pharmaceutical composition of the present invention, the specific radionuclide used, patient size and anatomy, the pharmacokinetics and biodistribution of the imaging agent. The predetermined time preferably results in an improvement in image quality due to increased detectability of the targeted tissue and reduced background noise. The predetermined time may be a predetermined time prior to intraoperative imaging. In some instances, the imaging could be dynamic, in which case the predetermined time may be as low as 0 hours. The predetermined time, e.g. prior to intraoperative imaging, may be at least 0 hours, at least 0.25 hours, at least 0.5 hours, or at least 1 hour. The predetermined time is preferably not more than 48 hours, more preferably not more than 24 hours, since after this time it is more likely that the tissue or body has excreted the compound, composition or pharmaceutical composition. The predetermined time, e.g. prior to intraoperative imaging is at least about 0.5 and preferably not more than about 48 hours. For example, the predetermined time may be at least about 1 (or about 2) and not more than about 36 hours; e.g. the predetermined time may be at least about 3 and not more than about 24 hours. The predetermined time is however not mandatory within the methods of the present invention.

In some embodiments, the composition of the present invention comprising a radiolabel may be detected via gamma-/beta-imaging and is useful for imaging in presurgical planning, intraoperative and postsurgical evaluation. The fluorophore may be detected by fluorescence imaging, an approach that provides a superior resolution and may be used to define the margins of tumors more clearly. This may be beneficial, for example during tumor excision surgery, as it may assist in guiding the removal of all diseased tissue, while preserving healthy tissue.

Moreover, in some embodiments, the invention relates to the use of the compound of formula (I), the composition or the pharmaceutical composition of the present invention as an analytical reference.

In further embodiments, the invention relates to the use of the compound of formula (I), the composition or the pharmaceutical composition of the present invention as an in vitro screening tool.

In some embodiments, the invention relates to a pharmaceutical composition comprising the compound of formula (I) according to any one of items 1 to 6, the composition of any one of items 7 to 9 or the pharmaceutical composition of item 10 for use in therapy, preferably for use in the treatment or prevention of a solid cancer, more preferably for use in the treatment or prevention of prostate cancer.

In some embodiments, the invention relates to the use of the compound of formula (I) according to any one of items 1 to 6, the composition of any one of items 7 to 9 or the pharmaceutical composition of item 10 for the manufacture of a medicament for the treatment or prevention of a disease, preferably wherein said disease is a solid cancer, more preferably wherein said disease is prostate cancer.

In some embodiments, the invention relates to a method of treating or preventing a disease/disorder in a subject, wherein the disease/disorder is a solid cancer, the method comprising administering the compound of formula (I) according to any one of claims 1 to 6, the composition of any one of claims 7 to 9 or the pharmaceutical composition of claim 10 to a subject in need thereof.

In some embodiments, the invention relates to the use of a compound of formula (I) according to any one of claims 1 to 6, the composition of any one of claims 7 to 9 or the pharmaceutical composition of claim 10 for the preparation of an agent for diagnosing a disorder/disease.

In some embodiments, the invention also provides a method of delivering a compound of formula (I) according to any one of claims 1 to 6, the composition of any one of claims 7 to 9 or the pharmaceutical composition of claim 10, the method comprising parenteral administration of said compound, composition or pharmaceutical composition.

In some embodiments, the invention relates to a kit comprising a compound of formula (I) according to any one of claims 1 to 6 and one or more radionuclides.

The following definitions apply throughout the present specification and the claims, unless specifically indicated otherwise.

The term “hydrocarbon group” refers to a group consisting of carbon atoms and hydrogen atoms.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₆ alkenyl” denotes an alkenyl group having 2 to 6 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₆ alkynyl” denotes an alkynyl group having 2 to 6 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₆ alkylene” denotes an alkylene group having 1 to 6 carbon atoms, and the term “C₀₋₆ alkylene” indicates that a covalent bond (corresponding to the option “Co alkylene”) or a C₁₋₆ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to a C₁₋₆ alkylene, C₁₋₅ alkylene, C₁₋₄ alkylene (including, in particular, a linear C₁₋₆ alkylene, C₁₋₅ alkylene, C₁₋₄ alkylene), a methylene or ethylene, most preferred is methylene.

As used herein, the term “cycloalkylene” refers to a divalent cyclic saturated hydrocarbon of three to eleven carbon atoms, including monocyclic rings as well as bridged ring, spiro ring and/or fused ring systems (which may be composed, e.g., of two or three rings; such as, e.g., a fused ring system composed of two or three fused rings). “Cycloalkylene” may, e.g., refer to cyclopropylene, cyclobutylene, cyclopentylene, cyclohexylene, cycloheptylene, decalinylene (i.e., decahydronaphthylene), or adamantylene. Unless defined otherwise, “cycloalkylene” refers to a C₃₋₁₁ cycloalkylene, and more preferably refers to a C₃₋₇ cycloalkylene. A particularly preferred “cycloalkylene” is a monocyclic saturated hydrocarbon ring having 3 to 7 ring members. Moreover, unless defined otherwise, the term “cycloalkylene” even more preferably refers to cyclohexylene or cyclopropylene, and yet even more preferably refers to cyclohexylene.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 1H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “halogen” refers to fluoro (—F), chloro (—CI), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A preferred “haloalkyl” group is fluoroalkyl. A particularly preferred “haloalkyl” group is —CF₃.

As used herein, the term “fluoroalkyl” refers to an alkyl group substituted with one or more (preferably 1 to 6, more preferably 1 to 3) fluoro atoms (—F). It will be understood that the maximum number of fluoro atoms is limited by the number of available attachment sites and, thus, depends on the number of carbon atoms comprised in the alkyl moiety of the fluoroalkyl group. “Fluoroalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂.

A particularly preferred “fluoroalkyl” group is —CF₃.

The terms “bond” and “covalent bond” are used herein synonymously, unless explicitly indicated otherwise or contradicted by context. The term “bond” may also refer to a dative covalent bond or coordinate bond, particularly when describing the interactions between a chelator and a metal such as in complexes. As used herein, a “coordinate bond” preferably refers to a shared pair of electrons between two atoms, wherein one atom supplies both electrons to the pair, e.g. wherein a nitrogen atom provides both electrons when bonded to a metal atom. As used herein, a “covalent bond” preferably refers to a shared pair of electrons between two atoms, wherein each atom supplies one electron to the pair, e.g. the bond between a carbon atom and hydrogen atom. As used herein, the terms “complex” and “chelate”, or grammatical variations thereof, may be used interchangeably to refer to chemical interactions of atoms through coordinate bonding.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to ±10% of the indicated numerical value, more preferably to ±5% of the indicated numerical value, and in particular to the exact numerical value indicated.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically acceptable salt forms of the compounds of formula (I), compositions or pharmaceutical compositions thereof which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I), composition or pharmaceutical composition thereof is a hydrochloride salt. Accordingly, it is preferred that the compound of formula (I), composition or pharmaceutical composition, including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I), composition or pharmaceutical composition is in the form of a hydrochloride salt.

The present invention also specifically relates to the compound of formula (I), including any one of the specific compounds of formula (I) described herein, in non-salt form.

Moreover, the scope of the invention embraces the compounds of formula (I), composition or pharmaceutical composition thereof in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I), compositions or pharmaceutical compositions of the invention are also encompassed within the scope of the invention. It is to be understood that such solvates and physical forms of pharmaceutically acceptable salts of the compounds of the formula (I), compositions or pharmaceutical compositions thereof are likewise embraced by the invention.

It will be appreciated that the compounds of formula (I) or the composition thereof may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). Complexes of any such isomers of the compounds of formula (I) are contemplated as being part of the present invention, either in admixture or in pure or substantially pure form. As for stereoisomers, the invention embraces isolated optical isomers of the compounds of formula (I) or compositions thereof as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I) and compositions thereof. It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I) or compositions thereof, in which one or more atoms are replaced by a specific isotope of the corresponding atom. For example, the invention encompasses a compound of formula (I) or the composition of the present invention, in which one or more hydrogen atoms (or, e.g., all hydrogen atoms) are replaced by deuterium atoms (i.e., ²H; also referred to as “D”). Accordingly, the invention also embraces complexes of a compound of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) or compositions thereof can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) or composition thereof is not enriched in deuterium. Accordingly, the presence of naturally occurring hydrogen atoms or ¹H hydrogen atoms in the compounds of formula (I) or compositions thereof is preferred. The present invention also embraces complexes of compounds of formula (I) or compositions thereof, in which one or more oxygen atoms (or, e.g., all oxygen atoms) are replaced by ¹⁷O atoms.

The compound of formula (I) or the composition provided herein may be administered as such or may be formulated as pharmaceutical compositions. The pharmaceutical compositions may optionally comprise one or more pharmaceutically acceptable excipients, such as bulking agents carriers, diluents, fillers, disintegrants, lubricating agents, binders, colorants, pigments, stabilizers, preservatives, antioxidants, and/or solubility enhancers. Preferred excipients are antioxidants and solubility enhancers. The diluent is preferably the buffer to be used for injection, which can, e.g., be a phosphate buffer. Furthermore, the diluent may include saccharides, including monosaccharides, disaccharides, polysaccharides and sugar alcohols such as arabinose, lactose, dextrose, sucrose, fructose, maltose, mannitol, erythritol, sorbitol, xylitol lactitol, and derivatives thereof.

Injectionable solutions of the compounds, compositions and complexes of the present invention can be formulated in saline or isotonic buffers, e.g., with a maximum ethanol content of 10%. A preferred example of a stabilizer is gentisinic acid (2,5-dihydroxybenzoic acid). Sodium ascorbate is preferably added as an antioxidant.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor© HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration.

The compound of formula (I) (or the corresponding composition or pharmaceutical compositions) may be administered to a subject by any convenient route of administration, whether systemically/peripherally or at the site of desired action, including but not limited to one or more of: an oral route (e.g., as a tablet, capsule, or as an ingestible solution); parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, (by, e.g., implant of a depot, for example, subcutaneously or intramuscularly), intraventricular, intraurethral, or intracranial route. A preferred route of administration is parenteral administration (particularly by injection techniques).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or the composition or pharmaceutical composition thereof can be administered alone (e.g., without concomitantly administering any therapeutic agents, or without concomitantly administering any therapeutic agents against the same disease that is to be imaged or diagnosed with the compounds of formula (I) or composition or pharmaceutical composition thereof). However, the compounds of formula (I) (or the corresponding composition or pharmaceutical compositions) can also be administered in combination with one or more therapeutic agents. If the compounds of formula (I), compositions or pharmaceutical compositions thereof is used in combination with a therapeutically active agent against the same disease or condition, the dose of each compound may differ from that when the corresponding compound is used alone, in particular, a lower dose of each compound may be used. The combination of the compounds of formula (I), compositions and pharmaceutical compositions thereof with one or more further therapeutic agents may comprise the simultaneous/concomitant administration of the compounds of formula (I), compositions or pharmaceutical compositions thereof and the therapeutic agent(s) (either in a single pharmaceutical formulation or in separate pharmaceutical formulations), or the sequential/separate administration of the compounds of formula (I) (or the corresponding composition or pharmaceutical compositions) and the further therapeutic agent(s). If administration is sequential, either the compounds of formula (I) (or the corresponding composition or pharmaceutical compositions) according to the invention or the one or more therapeutic agents may be administered first. If administration is simultaneous, the one or more therapeutic agents may be included in the same pharmaceutical formulation as the compounds of formula (I), compositions or pharmaceutical compositions thereof, or they may be administered in two or more different (separate) pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “imaging”, as used herein, refers to the imaging of a disorder or a disease, such as a solid cancer. In particular, the theranostic agent according to the present invention is particularly suited to imaging of a solid cancer by magnetic resonance imaging (MRI). As referred to herein, MRI includes and embraces magnetic resonance spectroscopic imaging as well as any other imaging modality or technique based on nuclear magnetic resonance.

The term “diagnosis”, as used herein, means confirmation of the presence or characteristics of a pathological condition. With regard to the present invention, diagnosis means confirmation of the presence of a solid cancer.

The term “solid cancer”, as used herein, refers to refers to one or more cells which are growing or have grown in an uncontrolled manner to form cancer tissue. As used herein, the term “solid cancer” includes, but is not limited to lung cancer, gastrointestinal cancer, colorectal cancer, colon cancer, anal cancer, liver cancer, pancreatic cancer, stomach cancer, genitourinary cancer, bladder cancer, biliary tract cancer, hepatobiliary cancer, testicular cancer, cervical cancer, ovarian cancer, uterine cancer, endometrial cancer, vaginal cancer, vulvar cancer, malignant mesothelioma, esophageal cancer, laryngeal cancer, prostate cancer, breast cancer, brain cancer, neuroblastoma, Ewing's sarcoma, osteogenic sarcoma, kidney cancer, epidermoid cancer, skin cancer, melanoma, head and/or neck cancer, mouth cancer, thymoma, Merkel-cell cancer, and neuroendocrine cancer, and particularly includes non-small cell lung cancer, colon cancer, and cancerous ovarian teratoma.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also illustrated by the appended figures.

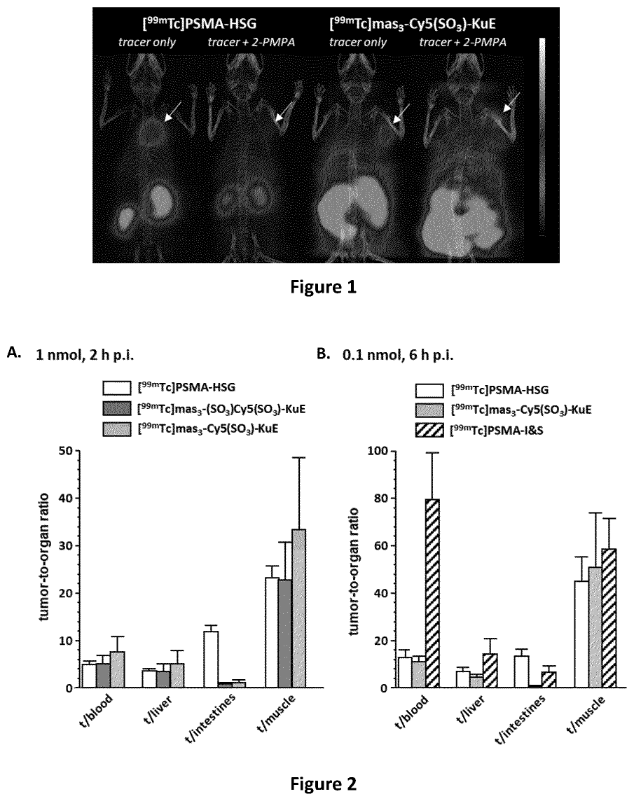

FIG. 1: Small animal SPECT/CT study in LNCaP xenograft bearing male NSG mice at 2-4 h p.i. Animals were either injected 24-34 MBq (constant ligand amount: 1 nmol) of [^{99m}Tc]PSMA-HSG only, or co-injected with 226 μg (1 μmol) 2-PMPA to demonstrate PSMA-specificity of tumor accumulation. White arrows indicate the position of the LNCaP xenograft.

FIG. 2: Tumor-to-organ ratios of the different ^{99m}Tc-labeled PSMA ligands investigated in this study. Data are means±SD (n=4-5 animals). A) Data obtained from the biodistribution study at 2 h p.i. using 1 peptide dose of 1 nmol/mouse; B) values at 6 h p.i. using a peptide dose of 0.1 nmol/mouse.

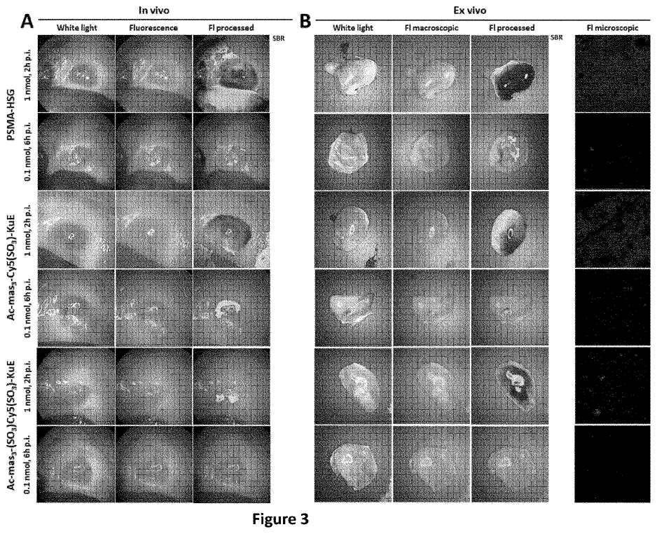

FIG. 3: In vivo (A) and ex vivo (B) fluorescence imaging of PC3-Pip xenografts in NSG mice using PSMA-HSG (upper two rows), the reference Ac-mas₃-Cy5(SO₃)—EuK (middle two rows) and Ac-mas₃-(SO₃)Cy5(SO₃)—EuK (lower two rows). Conditions were 2 h p.i. using a peptide dose of 1 nmol/mouse and 6 h p.i. using a peptide dose of 0.1 nmol/mouse, respectively (n=1 per condition). In vivo and ex vivo fluorescence imaging of intact tumors was performed using a clinical-grade fluorescence KARL STORZ Endoscope; fluorescence confocal imaging of 2 mm tumor slices (right) was performed using a Leica SP8 WLL microscope.

### EXAMPLES

The following compounds are referred to in the examples with the corresponding abbreviations or compound names (indicated above the structure). Where the compounds are a composition comprising the compound and a radionuclide, the specific radionuclide is indicated before the abbreviation or compound name, e.g. a composition comprising the compound PSMA-HSG and ^{99m}Tc is referred to as [^{99m}Tc]PSMA-HSG.

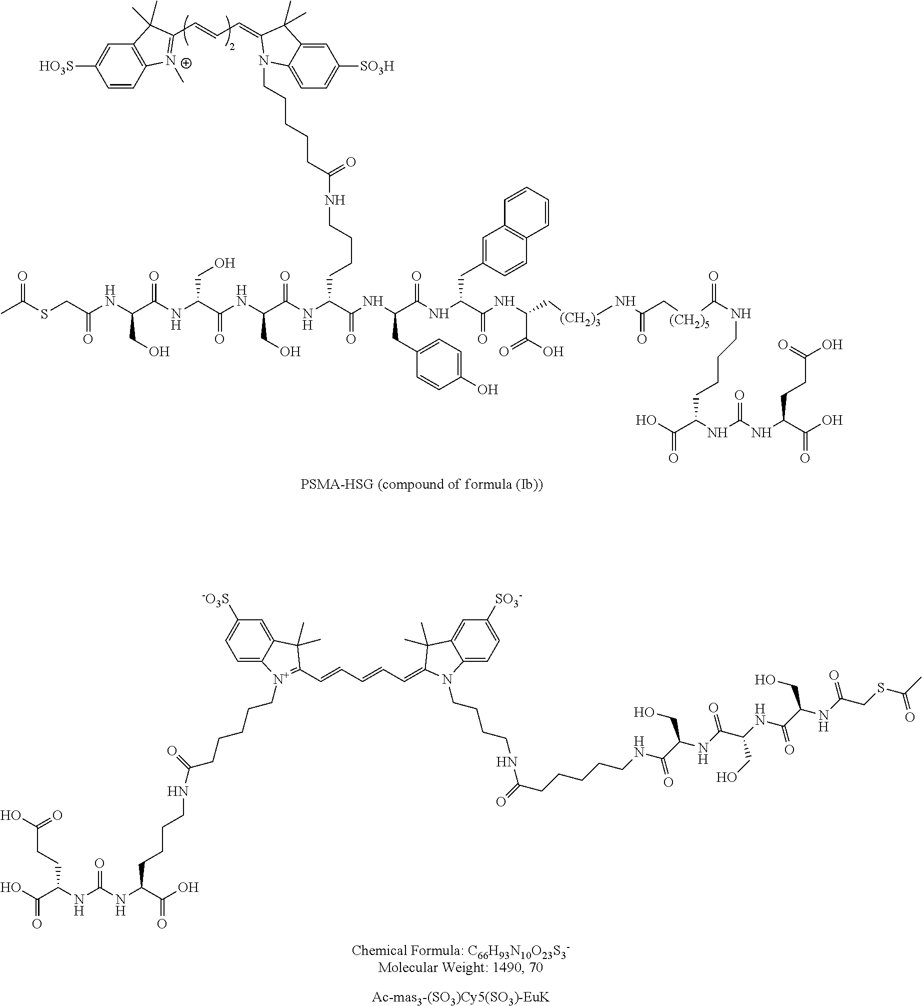

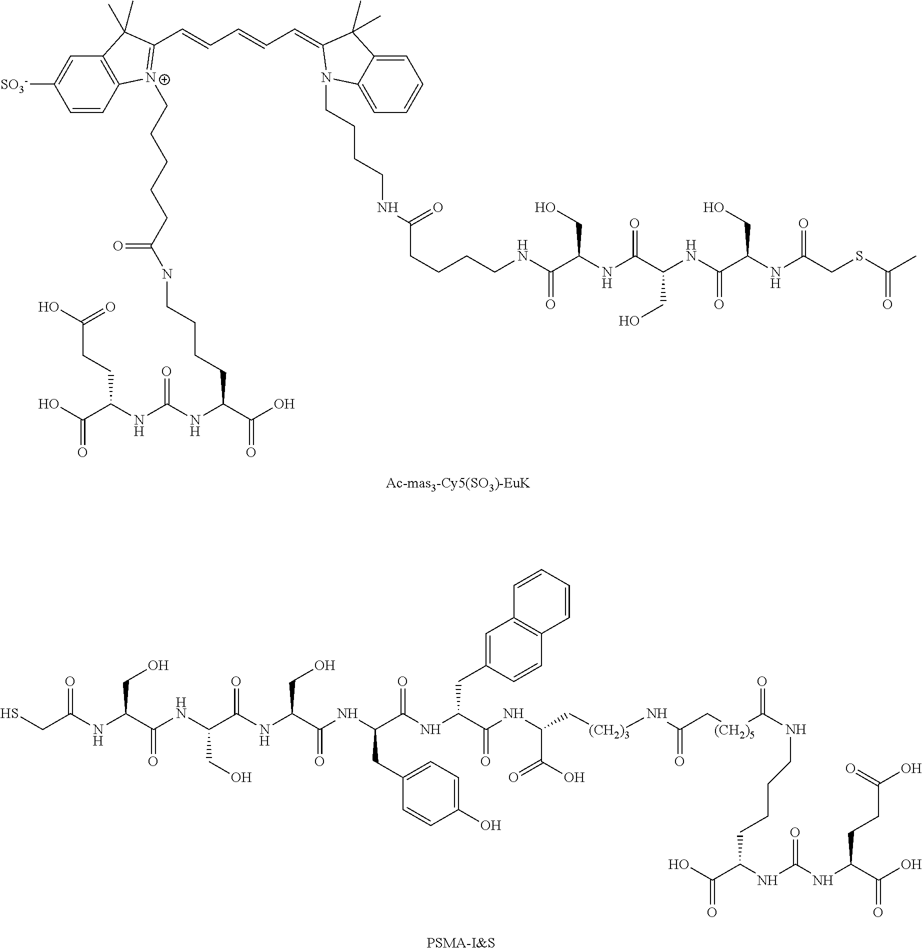

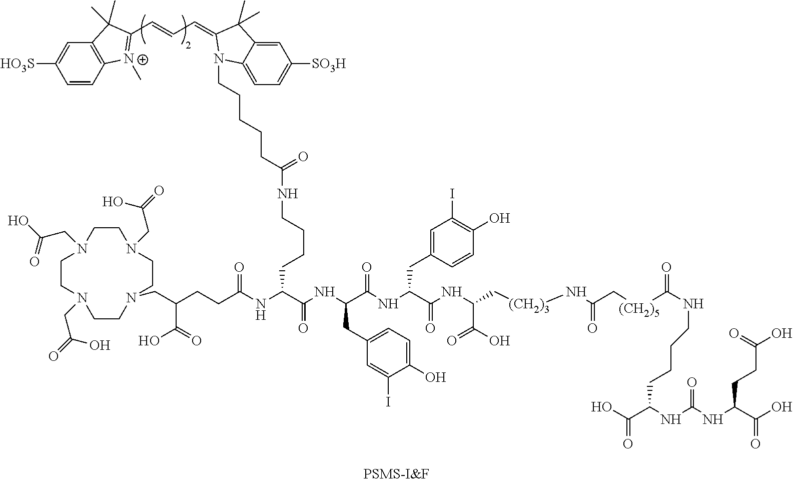

### Materials and Methods

Fmoc-(9-fluorenylmethoxycarbonyl-) and all other protected amino acid analogues as well as coupling reagents were purchased from Iris Biotech (Marktredwitz, Germany) or Bachem (Bubendorf, Switzerland). 2-Clorotritylchloride polystyrene (2-CTC) resin was obtained from Iris Biotech, Fmoc-Glu(OtBu)-loaded Wang resin was purchased from Novabiochem/Merck (Darmstadt, Germany). Solvents and all other organic reagents were obtained from Sigma-Aldrich (Munich, Germany), VWR (Dietikon, Switzerland) or Actu-AII (Oss, The Netherlands). Sulfo-Cy5 free acid was purchased from Lumiprobe (Hannover, Germany). Solid phase peptide synthesis (SPPS) was carried out manually.

Analytical reversed-phase high performance liquid chromatography (RP-HPLC) was performed on either a MultoKrom 100 C18 (5 μm, 125×4.0 mm) column (CS GmbH, Langerwehe, Germany), using a Shimadzu Corp. (Kyoto, Japan) system with a LC-40D gradient pump, a CBM-40 system controller, an CTO-40C column oven and a SPD-M40 UV/VIS photodiode array detector, or a Symmetry C18 (3.5 μm, 150×2.1) column (Waters, Massachusetts, USA) using a Waters Alliance 2690/2695 system and a 996 PDA. For semi-preparative HPLC, either a MultoKrom 100 RP 18 column (5 μm, 150×10 mm, CS Chromatographie GmbH, Langerwehe, Germany) with 4 mL/min flow rate or an XBridge BEH C8 OBD column (10 μm, 250×10 mm) with a 5 mL/min flow on a Waters 2545 with a Waters 2489 UV/Vis detector was used. Data analysis was performed using the Shimadzu Lab Solutions or Empower 2 software. Peptides were eluted applying different gradients of 0.1% (v/v) trifluoroacetic acid (TFA) in H₂O (solvent A) and 0.1% TFA (v/v) in acetonitrile (solvent B) at a constant flow of 1/0.3 mL/min (or 4-5 mL/min for semi-preparative HPLC); specific gradients are cited in the text. Retention times t_{R} are cited in the text. Electrospray ionization mass spectrometry (ESI-MS) was performed using an Advion expression CMS mass spectrometer (Advion, Harlow, UK). MALDI-TOF was performed using a Bruker Microflex (Massachusetts, USA). NMR was performed using a Bruker AV-300 (300 MHz) (Massachusetts, USA).

### Precursor Synthesis

The PSMA-HSG precursor backbone was synthesized using a novel, optimized solid-phase synthesis procedure. Only the final step, the conjugation with Sulfo-Cy5 free acid, using HOAt as the coupling reagent, was performed in solution phase. The synthesis of the reference compounds mas₃-(SO₃)Cy5-EuK and mas₃-(SO₃)Cy5(SO₃)—EuK has been described in detail elsewhere (see Hensbergen, A. W. et al., J. Nucl. Med., 2020, 61, 234-241).

### Synthesis of PSMA-HSG

### Synthesis of Building Blocks

### H-Lys(Dde)-OtBu

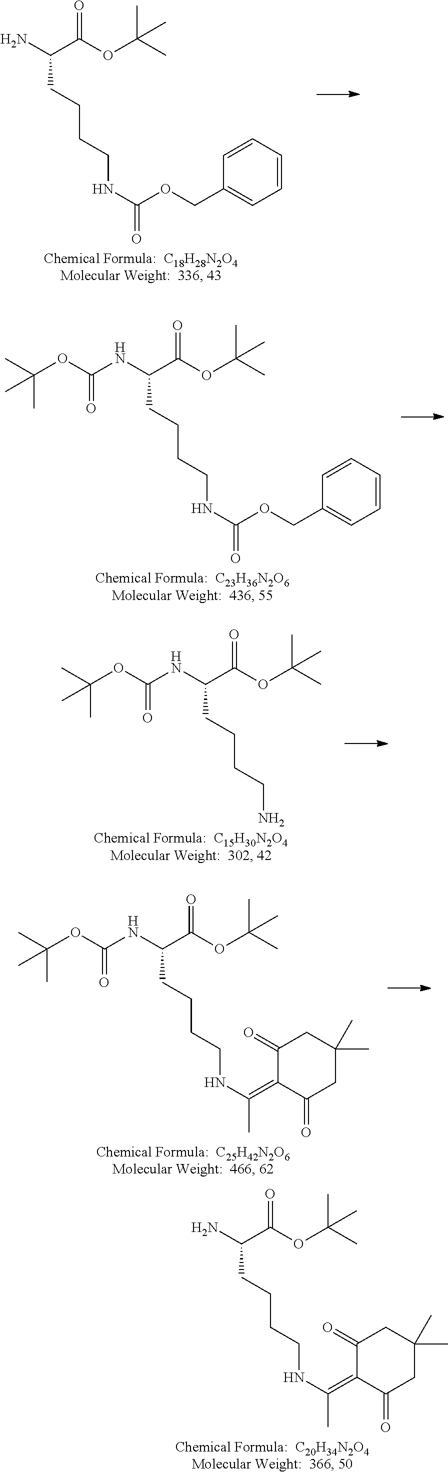 [SMILES: CC(C)(C)OC(=O)[C@@H](N)CCCCNC(=O)OCc1ccccc1 ; CC(C)(C)OC(=O)N[C@@H](CCCCNC(=O)OCc1ccccc1)C(=O)OC(C)(C)C ; CC(C)(C)OC(=O)N[C@@H](CCCCN)C(=O)OC(C)(C)C ; CC(NCCCC[C@H](NC(=O)OC(C)(C)C)C(=O)OC(C)(C)C)=C1C(=O)CC(C)(C)CC1=O ; CC(NCCCC[C@H](N)C(=O)OC(C)(C)C)=C1C(=O)CC(C)(C)CC1=O]

### Boc-Lys(Z)-OtBu (tButoxycarbonyl-Lysine(Z)-OtBu)

To a solution of H-Lys(Z)-OtBu·HCl (3.7 g, 10 mmol) in dichloromethane (DCM), Boc anhydride (3.3 g, 15 mmol) and triethylamine (TEA) (4.2 ml, 30 mmol) were successively added. After stirring overnight at room temperature (RT), analytical RP-HPLC revealed quantitative product formation. The DMC phase was extracted with water (3×), dried over MgSO₄ and evaporated to dryness to yield a colorless oil. HPLC (40-100%, 15 min): t_{R}=12.00 min.

### Boc-Lys-OtBu

The crude Boc-Lys(Z)-OtBu was dissolved in methanol (MeOH), and 440 mg (10% (w/w) based on protected amino acid) palladium on activated charcoal were added. The solution was stirred vigorously under H₂ pressure (balloon) for 24 h at RT. Completion of the deprotection reaction was confirmed using analytical RP-HPLC. The reaction mixture was passed through a syringe filter to remove the catalyst and evaporated to dryness, yielding crude Boc-Lys-OtBu as a clear oil in quantitative yield.

HPLC (40-100%, 15 min): t_{R}=9.85 min.

### Boc-Lys(Dde)-OtBu (Boc-Lys(2-Acetyldimedone)-OtBu)

For side-chain Dde-protection (2-Acetyldimedone-protection), crude Boc-Lys-OtBu was and Dde-OH (1.91 g, 1.05 eq.) were dissolved in DCM and stirred overnight. The solution was then evaporated to dryness, and upon purification via column chromatography (silica gel; ethyl acetate/petrol ether (bp 50-70° C.) 1:1 (v/v)), Boc-Lys(Dde)-OtBu was obtained in X % purity and 63% yield (based on H-Lys(Z)-OtBu starting material).

HPLC (40-100%, 15 min): t_{R}=10.83 min.

### H-Lys(Dde)-OtBu

For selective removal of the Boc protecting group, Boc-Lys(Dde)-OtBu was dissolved in 40 ml 1M TFA in acetonitrile and heated to 80° for 40 min in a sealed flask. Upon cooling to RT, the solvents were evaporated, and H-Lys(Dde)-OtBu was purified using column chromatography (silica gel; ethyl acetate/MeOH 95:5 (v/v)+0.5% (v/v) TEA). The desired product was obtained as a yellowish oil in 98% yield based on the protected precursor.

HPLC (10-90%, 15 min): t_{R}=8.26 min.

calculated molecular weight (C₂₀H₃₄N₂O₄): 366.5; found (ESI-MS): m/z=367.3 [M+H]⁺, 733.5 [2M+H]⁺

### Ac-mercaptoacetyl-D-Ser(tBu)-D-Ser(tBu)-D-Ser(tBu)-OH (Ac-mas₃(tBu)₃-OH)

The peptide sequence Fmoc-D-Ser(tBu)-D-Ser(tBu)-D-Ser(tBu)- was assembled manually on 2-CTC resin according to a standard Fmoc SPPS protocol using HOBt and TBTU as coupling reagents. Upon removal of the N-terminal Fmoc protecting group, solid phase coupling with S-Acetyl-thioglycolic acid was carried out by adding S-Acetyl-thioglycolic acid pentafluorophenyl ester (1.5 eq) and TEA (3 eq) in DMF. After shaking for 2 h at RT, the resin was washed with DMF and DCM, and the fully protected mas₃-chelator was cleaved from the resin using hexafluoroisopropanol in DCM (1:4 (v/v)). The product solution was evaporated to dryness, and upon lyophilization with tBuOH, the crude peptide chelator was obtained in 80% yield based on functional groups on the 2-CTC resin and X % purity (UV-detection at 214 nm).

HPLC (10-90%, 15 min): t_{R}=12.20 min,

calculated molecular weight (C₂₅H₄₅N₃O₉S): 563.7; found (ESI-MS): m/z=564.3 [M+H]⁺

### Fmoc Solid-Phase Peptide Synthesis of Ac-Mas3-k-v-Nal-k-Sub-KuE

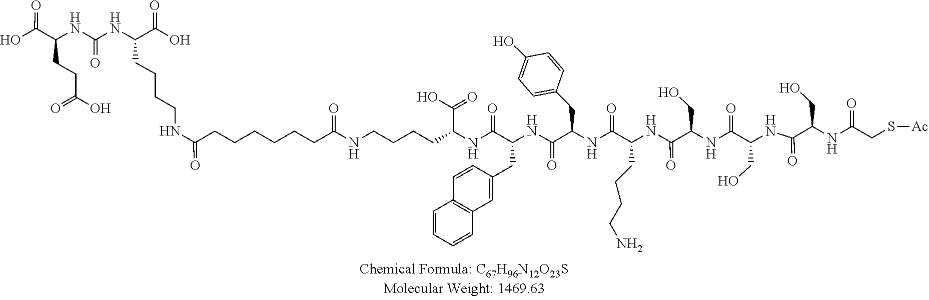 [SMILES: CC(=O)SCC(=O)N[C@H](CO)C(=O)N[C@H](CO)C(=O)N[C@H](CO)C(=O)N[C@H](CCCCN)C(=O)N[C@H](Cc1ccc(O)cc1)C(=O)N[C@H](Cc1ccc2ccccc2c1)C(=O)N[C@H](CCCCNC(=O)CCCCCCC(=O)NCCCC[C@H](NC(=O)N[C@@H](CCC(=O)O)C(=O)O)C(=O)O)C(=O)O]

Fmoc-Glu(OtBu)-preloaded Wang resin (500 mg, 0.33 mmol) was allowed to preswell in DMF for 30 min. After Fmoc-deprotection using 20% piperidine in DMF and washing with DMF (8×) and DCM (3×), the resin-bound H₂N-Glu(OtBu) was reacted overnight with 1,1′-Carbonyldiimidazole (1.1 eq), 4-(Dimethylamino)pyridine (0.04 eq) and TEA (2.5 eq) in DMF. After washing with DCM (6×), H-Lys(Dde)-OtBu (2 eq) and TEA (3 eq) in DCM were added to the resin, and reaction was allowed to proceed overnight. After washing with DCM (6×) and subsequent swelling in DMF for 30 min, Dde-deprotection was carried out using 2% hydrazine hydrate in DMF (v/v; 2×10 min). The resin was then washed with DMF (10×) and reacted overnight with Di-pentafluorophenyl suberate (2 eq) and Diisopropylethylamine (DIPEA, 1.5 eq) in DMF. Upon washing with DMF, the resin was reacted with Fmoc-D-Lys-OtBu (2 eq) and TEA (4 eq) in DMF for 1 h and washed with DMF. The subsequent coupling reactions with Fmoc-D-2-Nal-OH, Fmoc-D-Tyr(tBu)-OH and Fmoc-D-Lys(Boc)-OH were carried out according to a standard Fmoc SPPS protocol. For N-terminal functionalization with the fully protected mas₃-chelator, Ac-mas₃(tBu)₃-OH was first preactivated with Pentafluorophenol (1.5 eq), N,N′-Diisopropylcarbodiimide (1.5 eq) and Pyridine (2 eq) in DMF for 2 h. Then, the active ester solution was added to the resin that had previously been suspended in DMF containing 1 eq of TEA. After shaking for 2.5 h, the resin was washed with washed with DMF (8×) and DCM (3×), and the inhibitor-peptide construct was cleaved from the resin using TFA containing 2.5% (v/v) Triisopropylsilane. Upon removal of the majority of the solvent in vacuo, the crude product was precipitated using Diethyl ether, washed with ether and dried in vacuo. Preparative HPLC (20-60% B in 20 min) yielded 10.3 mg of Ac-mas₃-k-y-nal-k-Sub-KuE in >95% purity.

HPLC (20-60%, 15 min): t_{R}=7.19 min,

calculated molecular weight (C₆₇H₉₆N₁₂O₂₃S): 1468.6; found (ESI-MS): m/z=1469.5 [M+H]⁺, 1491.4 [M+Na]⁺, 735.4 [M+2H]²⁺.

### Conjugation with (SO₃)Cy5(SO₃)—COOH (Sulfo-Cy5 free acid) (PSMA-HSG)

Ac-mas₃-k-y-nal-k-Sub-KuE (6.8 μmol, 1 eq) was dissolved in 200 μL DMF, and 2 eq DIPEA were added. Subsequently, Sulfo-Cy5 free acid (1.1 eq) was preactivated with HATU (1.1 eq) and DIPEA (2 eq) in 200 μL DMF, and the mixture was added to the peptide precursor. Upon completion of the conjugation reaction (after 60 min), the crude product was precipitated from diethyl ether, washed and dried in vacuo. After preparative HPLC (25-40% B in 20 min), 1.74 mg Ac-mas₃-k(Sulfo-Cy5)-γ-nal-k-Sub-EuK (PSMA-HSG) were obtained in >95% purity (12% yield based on starting peptide).

HPLC (20-40%, 15 min): t_{R}=10.50 min,

calculated molecular weight (C₉₉H₁₃₃N₁₄O₃₀S₃⁺): 2095.4; found (ESI-MS): m/z=1048.3 [M+2H]²⁺.

### Synthesis of Ac-mas3-Cy5(SO₃)—EuK and Ac-mas3-(SO₃)Cy5(SO₃)—EuK

Both compounds were synthesized in analogy to a previously described protocol (Hensbergen, A. W., J. Nucl. Med., 2020; 61, 234-241).

### Synthesis of ((¹²⁵I)I-BA)EuK

The radioiodinated reference ligand ((¹²⁵I)I-BA)EuK ((S)-1-carboxy-5-(4-(-¹²⁵I-iodo-benzamido)pentyl)-carbamoyl)-L-glutamic acid) was prepared as described (Weineisen M. et al., Eur. J. Nuc. Mol. Med. Imaging Res., 2014, 4, 63).

### Photophysical Characterization

Quantum yield, molar extinction coefficient and serum protein binding experiments were performed according to previously published procedures (Hensbergen A. W. et al., Dyes and Pigments, 2018, 152, 19-28).

### Radiolabeling

For all compounds investigated in this study (PSMA-I&S, PSMA-HSG, Ac-mas₃-(SO₃)Cy5(SO₃)—EuK and Ac-mas₃-Cy5(SO₃)—EuK), kit-like reaction vials containing 5, 10 or 25 nmol of labelling precursor as well as fixed weight proportions of phosphate buffer, sodium tartrate, ascorbic acid, hydrochloric acid and stannous chloride dihydrate (SnCl₂·2H₂O) were prepared (Robu, S. et al., J. Nucl. Med., 2017, 58, 235-242), lyophilized and stored at −20° C. until use. For ^{99m}Tc-labeling, [^{99m}Tc]Tc-pertechnetate (usually 1 GBq) in saline (1.5-2 mL) was added to the sealed reaction vial, which was heated to 95° C. for 15 min. Upon cooling, the reaction mixture was diluted with deionized water to a total volume of 10 ml and passed through a SepPak C18 plus (Waters, Eschborn, Germany) cartridge (preconditioned with 5 ml ethanol and 5 ml deionized water). The cartridge was then washed with 6 ml of deionized water, and dried with air. For elution, 1 mL of ethanol (0.5% AcOH) was used, and fractions of 3-4 drops of eluate were collected in Eppendorf vials. The fractions containing the highest amount of activity were combined, evaporated to dryness at 80° C. under a nitrogen stream and reconstituted to the required activity concentration for the respective experiment using PBS.

Quality control of the final product was performed using Radio-TLC on silica-impregnated glass microfibre chromatography paper (Agilent, Basel, Switzerland), using two different mobile phases: 2-Butanone for determining the amount of free [^{99m}Tc]Tc-pertechnetate, and a 1:1 (v/v) mixture of 1M NaOAc/DMF to determine the amount of colloidal [^{99m}Tc]Tc-species. For all compounds, the overall radiochemical purity of the final product was always 95%.

Labeling with ^{99m}Tc was carried out using kit-like lyophilized reaction vials. When high specific activities were required (in vitro studies, biodistribution at 0.1 nmol peptide dose), reaction vials containing 5 or 10 nmol of labeling precursor were used. For SPECT/CT imaging and biodistribution studies at 1 nmol peptide dose, vials containing 25 nmol of labelling precursor were used. SPE purification and subsequent reconstitution in PBS provided all ^{99m}Tc-labeled ligands in >95% radiochemical purity (as determined by radio-TLC).

### Determination of Lipophilicity

The lipophilicity of [^{99m}Tc]PSMA-HSG, [^{99m}Tc]EuK(SO₃)Cy5-mas₃ and [^{99m}Tc]EuK(SO₃)Cy5(SO₃)-mas₃ was determined via a modified shake-flask method as described (Weineisen M. et al., Eur. J. Nuc. Mol. Med. Imaging Res., 2014, 4, 63).

### In Vitro Evaluation

Competitive binding experiments (IC₅₀) were carried out in accordance with an established protocol (Weineisen M. et al., Eur. J. Nuc. Mol. Med. Imaging Res., 2014, 4, 63) using PSMA-expressing LNCaP cells and ([¹²⁵I]I-BA)EuK as standard radioligand. Internalization kinetics of [^{99m}Tc]PSMA-HSG, [^{99m}Tc]EuK(SO₃)Cy5-mas₃ and [^{99m}Tc]EuK(SO₃)Cy5(SO₃)-mas₃ (0.5 nM) were investigated in dual tracer internalization assays using PSMA expressing LNCaP cells and ([¹²⁵I]I-BA)EuK (0.1 nM) as an internal reference.

### In Vivo Evaluation

All animal experiments were conducted in compliance with the Swiss legislation for care and use of laboratory animals under the license VD-3595.

### Biodistribution Studies

The biodistribution of [^{99m}Tc]PSMA-HSG, [^{99m}Tc]EuK(SO₃)Cy5-mas₃ and [^{99m}Tc]EuK(SO₃)Cy5(SO₃)-mas₃ was investigated in LNCaP xenograft bearing NSG mice. Mice were injected intravenously with the respective ^{99m}Tc-labeled tracers (24-34 MBq (fixed peptide dose of 1 nmol/mouse) or 3.7-7.6 MBq (fixed peptide doses of 0.1 nmol/mouse), respectively) and were sacrificed at 2 h (1 nmol-cohort) or 6 h p.i. (0.1 nmol-cohort), respectively. The organs of interest were dissected, and the activity concentration in weighed tissue samples was quantified using an AMG Automatic Gamma Counter (Hidex, Turku, Finland). Biodistribution data are given in % iD/g and represent means±SD (groups of n=5 animals).

### Small Animal SPECT/CT Imaging

SPECT/CT images were acquired using an Albira Si PET/SPECT/CT (Bruker Biospin Corporation, Woodbridge, CT, USA) instrument. Mice were injected with the respective ^{99m}Tc-labeled tracer (24-34 MBq; the injected peptide amount was kept constant at 1 nmol/animal), with (blocking) or without (control) co-injection of 226 μg (1 μmol) 2-PMPA (2-(Phosphonomethyl)-pentandioic acid). Mice were allowed to stay awake for 2 h and were then anesthetized for the duration of the imaging experiments by inhalation of 1.5% isoflurane/O₂ and placed on a heated bed (30-35° C.).

For SPECT imaging, static acquisitions of 2 h (accumulation of 1-2×10⁶ events) were acquired with the following parameters: photopeak at 140 keV±30%, axial FOV 82.5 mm using a single pinhole collimator. For reconstruction, an ordered subset expectation-maximization algorithm (OSEM) with two iterations was used, and scatter correction was applied. For CT, the following parameters were used: 400 μA intensity and 35 kV voltage, 600 projections. Images were reconstructed using a filtered back-projection (FBP) algorithm with de-ringing correction. Fused representative SPECT/CT images were acquired using the PMOD software (PMOD technologies, version 3.709, Zurich, Switzerland).

### In Vivo Fluorescence Imaging

In vivo fluorescence imaging of PC3-Pip xenografts in NSG mice (2 h p.i. at a ligand dose of 1 nmol and 6 h p.i. at a ligand dose of 0.1 nmol, respectively; n=1 per compound and condition) was performed using a clinical-grade fluorescence laparoscopy set-up (KARL STORZ Endoskope GMBh & Co. KG). Cy5-based tumor imaging was performed using D-light P system and a prototype modified IMAGE 1 S light source with integrated Cy5 filter (both KARL STORZ Endoskope GMBh & Co. KG). Images were recorded using integrated KARL STORZ software and image processing was performed using in-house developed MATLAB software that provided a signal intensity-based representation of the signal-to-background ratio (SBR; Azargoshasb S. et al., Mol. Imaging Biol., 2022, https://doi.org/10.1007/s11307-022-01736-y; de Barros H. A. et al., Eur. Urol., 2022, 82, 97-105).

Fluorescence Confocal Imaging of Excised Tumor Specimens After in vivo imaging tumor specimens (N=1 per tracer) were excised and cut into 2 mm slices. Prior to imaging, slices were placed on a 35-mm culture dish containing a glass insert (MatTek Co.). Fluorescence confocal imaging was then performed using a Leica SP8 WLL microscope (Leica Microsystems). For assessment of tracer uptake Cy5 settings were used; λₑₓ=633 nm, λₑₘ=650-700 nm. Images were processed using accompanying LASX software (Leica Application Software Suite 4.8).

### Results and Discussion

To set the data obtained with [^{99m}Tc]PSMA-HSG into the context of the recent developments in this field, we included two additional compounds into this study for comparative evaluation. In both compounds, [^{99m}Tc]mas₃-(SO₃)Cy5(SO₃)—EuK and [^{99m}Tc]mas₃-Cy5(SO₃)—EuK, the cyanine dye is employed as a linker unit (Hensbergen, A. W. et al., J. Nucl. Med., 2020, 61, 234-241). While [^{99m}Tc]mas₃-(SO₃)Cy5(SO₃)—EuK, containing the disulfonated Cy5-dye (as in PSMA-HSG) was included as a direct analog to [^{99m}Tc]PSMA-HSG (concerning the dye structure), the analog containing the mono-sulfonated Cy5 dye, [^{99m}Tc]mas₃-Cy5(SO₃)—EuK, was included as a “benchmark” hybrid ligand based on its previously established excellent in vivo performance. [^{99m}Tc]PSMA-I&S was also included into this comparative evaluation to be able to assess the influence of the dye conjugation on overall PSMA-targeting characteristics and in vivo behavior of [^{99m}Tc]PSMA-HSG.

### Photophysical Characterization

Table 1 summarizes the photophysical properties of [^{99m}Tc]PSMA-HSG, [^{99m}Tc]mas₃-Cy5(SO₃)—EuK and [^{99m}Tc]mas₃-(SO₃)Cy5(SO₃)—EuK and of the different sulfonated Cy5-dyes used for their preparation. As literature indicates that sulfonates not only play a role in the solubility, but also in the fluorescent quantum yield (Spa S. J. et al., Dyes and Pigments, 2018, 152, 19-28). Because of this latter effect we analyzed the brightness (as multiplication of the molar extinction coefficient and the quantum yield) in humans serum albumin for all three hybrid PSMA agents included in this study (see Table 1). While we indeed see that increasing of the number of sulfonates on the cyanine dye has a positive effect on the brightness all remain within the same order of magnitude.

TABLE 1
Photophysical properties and serum protein binding
of the PSMA ligands investigated in this study

| | Molar extinction | Quantum | Brightness |
|---|---|---|---|
| | coefficient free dye | yield | (HSA, L |
| Ligand | (HSA, L mol⁻¹cm⁻¹) | (HSA, %) | mol⁻¹cm⁻¹) |
| PSMA I&S | n.a. | n.a. | n.a. |
| PSMA-HSG | 161.600 | 26 | 42.020 |
| Ac-mas₃- | 160.900 | 21 | 33.790 |
| Cy5(SO₃)-EuK | | | |
| Ac-mas₃- | 243.000 | 16 | 38.880 |
| (SO3)Cy5(SO₃)-EuK | | | |

Literature indicates that sulfonates as substituents on Cy dyes not only play a role in solubility, but also in the fluorescent quantum yield (Hensbergen A. W. et al., Dyes and Pigments, 2018, 152, 19-28). Because of this latter effect, we analyzed the brightness (as multiplication of the molar extinction coefficient and the quantum yield) in human serum albumin solution for all three hybrid PSMA agents included in this study (see Table 1). While we indeed see that increasing of the number of sulfonates on the cyanine dye has a positive effect on the brightness, all remain within the same order of magnitude.

### In Vitro Evaluation

The in vitro characteristics of the two novel compounds [^{99m}Tc]PSMA-HSG and [^{99m}Tc]mas₃-(SO₃)Cy5(SO₃)—EuK are summarized in Table 2. Their in vitro profile was compared to that of the corresponding reference compounds, i.e. [¹²⁵I]IBA-EuK, [^{99m}Tc]PSMA-I&S, [⁶⁸GaPSMA-I&F and [^{99m}Tc]mas₃-Cy5(SO₃)—EuK, all of which had been assayed under identical experimental conditions.

It was found that PSMA-HSG shows improved PSMA-affinity compared to PSMA-I&S (Robu S. et al., J. Nucl. Med., 2017, 58, 235-242). In contrast, Ac-mas₃-(SO₃)Cy5(SO₃)—EuK, where the sulfonated Cy5 dye acts as linker between EuK and Ac-mas₃ shows an almost eight-fold reduction in affinity compared to the mono-sulfonated analog Ac-mas₃-Cy5(SO₃)—EuK (Table 2). This effect is clearly related to the interaction between the charged dye and the remote arene binding site in PSMA (Zhang, A. X. et al., J. Am. Chem. Soc., 2010, 132, 12711-12716) and a result of the compact design of the dye-bridged ligands, using no additional linker between the dye and the binding motif. The introduction of a linker, however, may help to overcome this limitation. Thus, with regard to PSMA binding affinity, although both linear (dye-bridged) and branched (dye end-label) ligand designs provide high-affinity binders (PSMA-HSG and Ac-mas₃-Cy5(SO₃)—EuK, Table 2), the branched design of PSMA-HSG is less sensitive towards the integration of disulfonated Cy5.

TABLE 2
PSMA affinities and lipophilicities (log D) of the novel hybrid ligand and selected
reference compounds (Robu, S. et al., J. Nucl. Med., 2017, 58, 235-242; Schottelius, M. et al.,
J. Nucl. Med., 2019, 60, 71-78; Hensbergen, A. W. et al., J. Nucl. Med., 2020, 61, 234-241).

| | IC₅₀ | | lipophilicity | Plasma Protein |
|---|---|---|---|---|
| Ligand | [nM] | Tracer | [log P_{OW}] | Binding (%) |
| (IBA)EuK | 7.1 ± 2.4 | ([¹²⁵I]IBA)EuK | −2.41 | n.d. |
| PSMA-I&S^{#} | 39.7 ± 1.2 | [^{99m}Tc]PSMA-I&S | −3.01 | 96.6 |
| [Ga]PSMA-I&F | 10.5 ± 2.1 | [⁶⁸Ga]PSMA-I&F | −3.40 | 94.0 |
| PSMA-HSG^{#} | 29.6 ± 5.6 | [^{99m}Tc]PSMA-HSG | −2.94 | 99.3 |
| Ac-mas₃-Cy5(SO₃)-EuK^{#} | 17.8 ± 2.9 | [^{99m}Tc]mas₃-Cy5(SO₃)-EuK | −1.94 | 96.5 |
| Ac-mas₃-(SO₃)Cy5(SO₃)-EuK^{#} | 131 ± 29 | [^{99m}Tc]mas₃-(SO₃)Cy5(SO₃)-EuK | −2.99 | 74.4 |

^{#}free mas₃-chelator

### Lipophilicity and Plasma Protein Binding

Both lipophilicity (log D) and plasma protein binding (PPB) are key physicochemical properties that drive the excretion pathway and general pharmacokinetics of a tracer. For structurally closely related compounds, these two parameters may correlate. However, the extent to which log D can represent the interactions of tracers with plasma proteins is highly variable and structure-dependent (Zoghbi S. S. et al., J. Pharm. Sci., 2012; 101, 1028-39.). Throughout the literature, however, the log D represents a robust indicator for the favored excretion pathway of a tracer. However, net charge, charge distribution, the radiolabeling method and tracer metabolic stability have a major impact on tracer handling and retention in the respective excretion organs (Gotthardt M. et al., J. Nucl. Med., 2007; 48, 596-601; Hosseinimehr S. J. et al., Drug Discov. Today, 2012, 17, 1224-32).

Plasma protein binding (PPB), in turn, has a decisive impact on tracer pharmacokinetics and bioavailability and thus also modulates tracer excretion. Compounds with a high tendency to associate with plasma proteins tend to show a longer blood retention and consequently increased uptake in well-perfused organs such as the liver, while compounds with lower binding are more rapidly cleared via the kidneys. In recent years, albumin binders such as Evans blue, 4-(p-iodo)phenyl butyric acid, ibuprofen or fatty acids have been conjugated to radiotracers to increase tracer uptake in the target (Lau J. et al, Bioconjug. Chem., 2019, 30, 487-502). Accordingly, fluorescent-dye-induced albumin binding also increases the background uptake (Bunschoten A. et al., Bioconjug. Chem., 2016, 27, 1253-8).

Of the four tracers under comparison for this study, all except [^{99m}Tc]mas₃-(SO₃)Cy5(SO₃)—EuK (74.4%) seem to be strong serum binders, with [^{99m}Tc]PSMA-HSG (99.3%) showing the highest PPB, closely followed by [^{99m}Tc]PSMA-I&S (96.6%) and [^{99m}Tc]mas₃-Cy5(SO₃)—EuK (96.5%). Interestingly, the differences between [^{99m}Tc]mas₃-(SO₃)Cy5(SO₃)—EuK and [^{99m}Tc]mas₃-Cy5(SO₃)—EuK (Table 2) correlate with the log D of the compounds and clearly reflect the effect of the additional sulfonate group in [^{99m}Tc]mas₃-(SO₃)Cy5(SO₃)—EuK. For the branched-design compounds [⁶⁸Ga]PSMA-I&F, [^{99m}Tc]PSMA-I&S and [^{99m}Tc]PSMA-HSG, this correlation does not seem to hold. Rather, PPB of these compounds seems to be dominated by the lipophilic linkers used in these compounds, and to a lesser degree by the radiolabeling method used.

### In Vivo Biodistribution and Small Animal SPECT/Fluorescence Imaging

To assess the influence of the different ligand designs of [^{99m}Tc]PSMA-HSG and [^{99m}Tc]mas₃-(SO₃)Cy5(SO₃)—EuK on in vivo PSMA-targeting and general pharmacokinetics, their in vivo biodistribution was investigated in LNCaP xenograft bearing NSG mice in two different experimental settings:
A) at 2 h p.i. using a ligand dose of 1 nmol/animal. These conditions were chosen to allow comparability of the data from this study (Table 3) with previous results in a different tumor model (Hensbergen, A. W. et al., J. Nucl. Med., 2020, 61, 234-241). Here, [^{99m}Tc]mas₃-Cy5(SO₃)—EuK was additionally included as an internal reference to establish the “missing link” between studies.
B) at 6 h p.i. using a ligand dose of 0.1 nmol/animal (Table 4). The latter settings were adapted to a timeline and dosing with more similarity to the clinical situation (surgery at a later time point, microdosing principle). Of note, however, a ligand amount of 0.1 nmol/mouse (extrapolation: 280 nmol/70 kg patient) still exceeds the potentially applicable clinical dose (48 nmol/70 kg patient for a compound with a molecular weight of −2100 g/mol). Nevertheless, it represents a dose level at which significant competition effects between unlabeled precursor and radioligand for PSMA binding sites in tumor tissue are not probable (Eder A. C. et al., J. Nucl. Med., 2021, 62, 1461-1467; Kalidindi T. M. et al., Eur. J. Nucl. Med. Mol. Imaging, 2021, 48, 2642-2651).

TABLE 3
Biodistribution of [^{99m}Tc]PSMA-HSG, [^{99m}Tc]mas₃-
Cy5(SO₃)-EuK and [^{99m}Tc]mas₃-(SO₃)Cy5(SO₃)-EuK in
LNCaP xenograft bearing male NSG mice (n = 4-5) at 2 h
p.i.. Mice were injected with a fixed ligand dose of 1
nmol/animal. Data are given in % iD/g and are means ± SD.

| | [^{99m}Tc]PSMA- | [^{99m}Tc]mas₃- | [^{99m}Tc]mas₃- |
|---|---|---|---|
| organ | HSG | (SO₃)Cy5-EuK | (SO₃)Cy5(SO₃)-EuK |
| blood | 2.4 ± 0.3 | 0.9 ± 0.1 | 0.7 ± 0.1 |
| heart | 1.3 ± 0.2 | 0.4 ± 0.1 | 0.3± 0.1 |
| lung | 2.8 ± 0.3 | 1.1 ± 0.1 | 1.1 ± 0.2 |
| liver | 3.3 ± 0.3 | 1.3 ± 0.5 | 1.0 ± 0.4 |
| spleen | 5.2 ± 1.9 | 1.9 ± 0.5 | 4.3 ± 1.3 |
| pancreas | 0.8 ± 0.1 | 0.3 ± 0.1 | 0.3 ± 0.1 |
| stomach | 1.6 ± 0.1 | 0.5 ± 0.1 | 0.6 ± 0.1 |
| intestines | 1.0 ± 0.1 | 6.3 ± 2.5 | 4.2 ± 0.4 |
| kidneys | 61.7 ± 7.2 | 21.0 ± 4.2 | 59.2 ± 14.4 |
| salivary glands | 4.2 ± 0.5 | 1.7 ± 0.3 | 3.1 ± 0.5 |
| muscle | 0.5 ± 0.04 | 0.2 ± 0.03 | 0.2 ± 0.03 |
| LNCaP tumor | 12.1 ± 0.9 | 6.6 ± 2.8 | 3.5 ± 1.1 |

The particularly pronounced tendency of [^{99m}Tc]PSMA-HSG to associate with plasma proteins (99.4% vs 77.9% for Ac-mas₃-(SO₃)Cy5(SO₃)—EuK and 97.2% for Ac-mas₃Cy5(SO₃)—EuK, Table 1), results in its particularly slow blood clearance, leading to elevated overall background activity levels at both time points and dose levels (Tables 3 and 4). This enhanced bioavailability for a prolonged time, however, also leads to high accumulation of [^{99m}Tc]PSMA-HSG in the LNCaP xenografts as well as PSMA-positive tissues such as lung, salivary glands and kidneys at both time points investigated. That the tracer uptake in these tissues is indeed PSMA-specific was confirmed via a SPECT/CT blocking study (FIG. 1).

Compared to [^{99m}Tc]PSMA-HSG, the doubly sulfonated analog [^{99m}Tc]mas₃-(SO₃)Cy5(SO₃)—EuK shows substantially faster clearance from the circulation and thus much lower general background uptake (Table 3). In accordance with its faster blood clearance and a 4.5-fold lower PSMA-affinity, [^{99m}Tc]mas₃-(SO₃)Cy5(SO₃)—EuK displays markedly reduced tumor accumulation compared to [^{99m}Tc]PSMA-HSG. In contrast, its uptake in other PSMA-positive tissues (salivary glands, kidney) as well as in spleen is similar to the uptake pattern of [^{99m}Tc]PSMA-HSG, despite the differences in PSMA affinity. This hints strongly towards a charge-dependent accumulation of both [^{99m}Tc]PSMA-HSG and [^{99m}Tc]mas₃-(SO₃)Cy5(SO₃)—EuK in these tissues. To date, the exact uptake mechanism for PSMA-targeted imaging probes into the salivary glands is still not fully understood, but has been shown to have a significant non-PSMA-mediated component (Heynickx N. et al., Nucl. Med. Biol., 2021, 98-99, 30-39). However, for the kidneys, a clear correlation between the number of sulfonates on the cyanine dye with renal retention has been established for hybrid RGD peptides (Bunschoten A., Bioconjug. Chem., 2016, 27, 1253-1258). This and the threefold lower kidney accumulation of the reference compound [^{99m}Tc]mas₃-Cy5(SO₃)—EuK (Table 3) compared to both ligands containing disulfo-Cy5 corroborates the above hypothesis.

Besides the differences in tumor accumulation, the most striking difference between [^{99m}Tc]PSMA-HSG and [^{99m}Tc]mas₃-(SO₃)Cy5(SO₃)—EuK is the much higher degree of hepatobiliary clearance and subsequent intestinal accumulation of the dye-bridged compound (Table 3). Based on the nearly identical lipophilicities of [^{99m}Tc]PSMA-HSG and [^{99m}Tc]mas₃-(SO₃)Cy5(SO₃)—EuK, differences in physicochemical characteristics of the two compounds cannot account for the distinct elimination patterns. Thus, the observed differences hint towards a structure-related effect, with the preferential intestinal uptake of [^{99m}Tc]mas₃-(SO₃)Cy5(SO₃)—EuK being a direct result of the dye positioning in the molecule.

This is further supported by the very similar general biodistribution patterns of [^{99m}Tc]mas₃-(SO₃)Cy5(SO₃)—EuK and the mono-sulfonated reference compound [^{99m}Tc]mas₃-Cy5(SO₃)—EuK (Table 3). With only one charged sulfonate on the bridging dye, [^{99m}Tc]mas₃-Cy5(SO₃)—EuK is more lipophilic (Table 2) and thus shows higher absolute intestinal uptake than its disulfonated-analog. Furthermore, its tumor uptake is enhanced as a result of higher PSMA-affinity (Table 2). However, in the direct comparative study, there is clear evidence for structure-related differences in biodistribution between the branched [^{99m}Tc]PSMA-HSG and the dye-bridged analogs [^{99m}Tc]mas₃-(SO₃)Cy5(SO₃)—EuK and [^{99m}Tc]mas₃-Cy5(SO₃)—EuK.

Of note, these differences and patterns would not have been as clearly distinguishable by using previous data on [^{99m}Tc]mas₃-Cy5(SO₃)—EuK as a reference (Hensbergen, A.

W. et al., J. Nucl. Med., 2020, 61, 234-241). In another mouse model (PC346C orthotopic xenografts in Balb/c nude mice, 1 nmol ligand, 2 h p.i.), the tumor accumulation of [^{99m}Tc]mas₃-Cy5(SO₃)—EuK was significantly higher (15.2±2.9% iD/g), whereas absolute intestinal uptake was lower than in the present study (1.61±0.29% iD/g). Furthermore, nonspecific background activity uptake was by a factor of 2 lower in the Balb/c model, while kidney uptake was identical to the values found in this study. This highlights the importance of side-by-side comparison of tracers in the same mouse model for valid comparisons.

Surprisingly, despite the fundamental differences in absolute tracer uptake observed for [^{99m}Tc]PSMA-HSG and [^{99m}Tc]mas₃-(SO₃)Cy5(SO₃)—EuK, the tumor/background ratios and at 2 h p.i. and 1 nmol ligand dose are nearly identical (with the exception of tumor/intestines, FIG. 2). This, can be explained by the substantially faster background clearance of [^{99m}Tc]mas₃-(SO₃)Cy5(SO₃)—EuK and the resulting lower background activity levels in all organs (except intestines), which numerically “compensate” for the lower absolute tumor uptake of the compound (Table 3).

However, for the intended application of the compounds in this study, i.e. radio- and fluorescence guided surgery of PSMA over-expressing cancer lesions, absolute ligand uptake is of particular importance for ensuring sufficient fluorescence intensity in tumor lesions during surgery. To validate this hypothesis, a comparative in vivo and ex vivo fluorescence imaging study was performed with the three ligands in this study, PSMA-HSG, Ac-mas3-(SO₃)Cy5(SO₃)—EuK and Ac-mas₃-Cy5(SO₃)—EuK in a PC3-Pip tumor model.

Both the in vivo and ex vivo fluorescence imaging results (FIG. 3) confirm the relevance of absolute tumor accumulation of fluorescence/hybrid PSMA ligands with comparable tumor/background ratios for the sensitivity of fluorescence detection in a surgical setting. At a ligand dose of 1 nmol (2 h p.i.), the relative fluorescence intensity detected with a clinical-grade endoscope for PSMA-HSG, Ac-mas₃-(SO₃)Cy5(SO₃)—EuK and Ac-mas₃-Cy5(SO₃)—EuK was found to clearly correlate with the tumor accumulation of the corresponding ^{99m}Tc-labeled tracers (Table 3). Both in the in vivo (Panel A, FIG. 3) and in the ex vivo setting (Panel B, FIG. 3, both macroscopic and microscopic images), PSMA-HSG with its particularly high tumor accumulation provided the most intense fluorescence signal in the xenografts, followed by Ac-mas₃-Cy5(SO₃)—EuK and Ac-mas₃-(SO₃)Cy5(SO₃)—EuK, which provided the weakest signal, as confirmed by confocal microscopy. It is important to note, however, that the fluorescence signal observed in this experiment is not only a function of tumor accumulation, but also correlates with the relative brightness of the compounds investigated (Table 1). The striking difference between the fluorescence imaging results obtained for the two di-sulfonated analogs PSMA-HSG and Ac-mas₃-(SO₃)Cy5(SO₃)—EuK are therefore a combined effect of a high tumor uptake with particularly high brightness (PSMA-HSG) on the one hand as opposed to comparably low tumor uptake and fivefold lower brightness (Ac-mas₃-(SO₃)Cy5(SO₃)—EuK) on the other hand. Both extremes are clearly related to the relative positioning of the disulfo-Cy5 dye within the molecule, with a triple combined effect on photophysical properties (Table 1), PSMA-targeting (Table 2) and pharmacokinetics (Table 3), respectively.

As shown in FIG. 3, fluorescence imaging experiments with PSMA-HSG, Ac-mas₃-(SO₃)Cy5(SO₃)—EuK and Ac-mas₃-Cy5(SO₃)—EuK were also carried out under a second set of experimental conditions, i.e. at 6 h p.i. using 0.1 nmol ligand, respectively.

As mentioned previously, these conditions were chosen such as to be more close to the clinical setting (late time point of radio/fluorescence guided surgery, microdosing principle). Under these conditions, the detectable fluorescence signal was weak for PSMA-HSG and the mono-sulfonated reference Ac-mas₃-Cy5(SO₃)—EuK, especially in the in vivo setting, but virtually not detectable for Ac-mas₃-(SO₃)Cy5(SO₃)—EuK.

Thus, this compound was not included in the subsequent comparative biodistribution study under these conditions (6 h p.i., 0.1 nmol ligand). In contrast, to be able to assess the influence of Sulfo-Cy5 conjugation on the pharmacokinetics of [^{99m}Tc]PSMA-HSG, the ligand [^{99m}Tc]PSMA-I&S was included at this point. Furthermore, to provide a link to the in vivo characteristics of the tracers based on the linear, integrated design, [^{99m}Tc]mas₃-Cy5(SO₃)—EuK. was also included as a reference.

Recent studies have already demonstrated progressive blocking of endogenous PSMA binding sites as a result of decreasing specific activity of the respective radioligand (Eder A. C. et al., J. Nucl. Med., 2021, 62, 1461-1467; Kalidindi T. M. et al., Eur. J.

Nucl. Med. Mol. Imaging, 2021, 48, 2642-2651; Soeda F. et al., J. Nucl. Med., 2019, 60, 1594-1599). Higher ligand doses (at constant injected activity) invariably led to reduced uptake of PSMA-targeted tracers in tissues with low (compared to tumor xenografts) endogenous PSMA expression, e.g. kidney or salivary glands. Of note, as discussed above, this effect may not only be caused by blocking of specific, PSMA-mediated tissue accumulation. Higher ligand doses may also lead to a progressive inhibition of non-specific, charge-dependent tissue uptake.

However, independent of the exact underlying mechanism, this effect was also observed for the tracers in this study when comparing the data obtained for a tenfold lower tracer dose (at 6 h p.i., Table 4) with the initial experimental settings (Table 3).

TABLE 4
Biodistribution of [^{99m}Tc]PSMA-HSG, [^{99m}Tc]PSMA-
I&S and [^{99m}Tc]mas₃-Cy5(SO₃)-EuK in LNCaP xenograft
bearing male NSG mice (n = 5) at 6 h p.i.. Mice
were injected with a fixed ligand dose of 0.1 nmol/animal.
Data are given in % iD/g and are means ± SD.

| | [^{99m}Tc]PSMA- | [^{99m}Tc]PSMA- | [^{99m}Tc]mas₃- |
|---|---|---|---|
| organ | HSG | I&S | Cy5(SO₃)- EuK |
| blood | 1.3 ± 0.3 | 0.15 ± 0.03 | 0.7 ± 0.1 |
| heart | 0.7 ± 0.2 | 0.24 ± 0.05 | 0.3 ± 0.1 |
| lung | 2.5 ± 0.5 | 2.2 ± 0.2 | 0.8 ± 0.2 |
| liver | 2.5 ± 0.4 | 0.8 ± 0.4 | 1.7 ± 0.3 |
| spleen | 9.7 ± 3.8 | 14.2 ± 2.0 | 1.9 ± 0.6 |
| pancreas | 0.8 ± 0.3 | 0.7 ± 0.1 | 0.3 ± 0.1 |
| stomach | 1.1 ± 0.2 | 0.3 ± 0.1 | 1.4 ± 0.3 |
| intestines | 1.3 ± 0.2 | 1.8 ± 0.8 | 9.7 ± 1.1 |
| kidneys | 148.1 ± 17 | 147.0 ± 10 | 70.0 ± 22 |
| salivary glands | 4.0 ± 1.2 | 3.2 ± 0.8 | 8.9 ± 2.1 |
| muscle | 0.4 ± 0.06 | 0.2 ± 0.04 | 0.2 ± 0.1 |
| LNCaP tumor | 17.1 ± 2.9 | 11.6 ± 1.4 | 8.1 ± 1.4 |

Both for [^{99m}Tc]PSMA-HSG and [^{99m}Tc]mas₃-Cy5(SO₃)—EuK, accumulation in the PSMA-positive kidneys was substantially higher at the 0.1 nmol dose. At the same time, as anticipated from the slow clearance kinetics of [^{99m}Tc]PSMA-HSG, its tumor accumulation increased by 50% over time. Due to its faster clearance and lower bioavailability at later time points, the tumor uptake of [^{99m}Tc]mas₃-Cy5(SO₃)—EuK was only slightly increased (Tables 3 and 4). Overall, tumor/kidney ratios were markedly decreased at the lower ligand dose (0.12±0.02 and 0.12±0.04 for [^{99m}Tc]PSMA-HSG and [^{99m}Tc]mas₃-Cy5(SO₃)—EuK, respectively, vs 0.20±0.03 and 0.31±0.15 at 2 h p.i./1 nmol). This is indicative of the anticipated blocking effect of the high ligand dose (1 nmol, Table 3) on renal tracer accumulation (Kalidindi T. M. et al., Eur. J. Nucl. Med. Mol. Imaging, 2021, 48, 2642-2651) and suggests that the biodistribution data obtained for [^{99m}Tc]PSMA-HSG and [^{99m}Tc]mas₃-Cy5(SO₃)—EuK under the low-dose/late-time-point conditions are more likely to be predictive for the human situation, given the micro-dosing principle is applied.

The direct side-by-side comparison of [^{99m}Tc]PSMA-HSG and its non-fluorescent parent peptide [^{99m}Tc]PSMA-I&S shows similar overall biodistribution patterns, with the only major difference being the substantially slower blood clearance of the hybrid analog, leading to overall higher background activity levels at 6 h p.i. (Table 4). This, alongside with an improved PSMA-affinity, leads to a 50% enhanced tumor accumulation for [^{99m}Tc]PSMA-HSG compared to [^{99m}Tc]PSMA-I&S, whereas kidney uptake remains unchanged, and intestinal activity accumulation is even reduced, due to the higher hydrophilicity of the Sulfo-Cy5-conjugate. Again, this seems to be due to the use of the extended linker in PSMA-HSG, which places the dye moiety at a very remote position from the binding motif. This minimizes its influence on binding affinity and, in this specific case, even allows exploiting the molecular characteristics of the dye as a beneficial “pharmacokinetic modifier”.

In contrast, the other compounds in this study, in which the dye constitutes part of the linker and interacts with the binding pocket of PSMA, inherently show much greater sensitivity towards even minor modifications. As shown, the position of the sulfonate group in [^{99m}Tc]mas₃-Cy5(SO₃)—EuK vs [^{99m}Tc]mas₃-(SO₃)Cy5-EuK already had a major impact on PSMA targeting efficiency. Consequently, the introduction of the additional sulfonate group in [^{99m}Tc]mas₃-(SO₃)Cy5(SO₃)—EuK lead to substantially reduced PSMA-affinity and tracer uptake in tumor xenografts (Tables 1 and 3), while, surprisingly, only minimally affecting overall tracer pharmacokinetics compared to [^{99m}Tc]mas3-Cy5(SO₃)—EuK (Table 3).

Thus, in summary, in this very specific side-by-side comparison of [^{99m}Tc]PSMA-HSG and [^{99m}Tc]mas₃-(SO₃)Cy5(SO₃)—EuK, the branched design of [^{99m}Tc]PSMA-HSG is much more robust towards integration of the di-sulfonated Cy5 dye and shows superior characteristics on all levels (photophysical, PSMA-affinity, in vivo PSMA-targeting, pharmacokinetics, fluorescence imaging).

In summary, the major asset of [^{99m}Tc]PSMA-HSG is its long circulation time and its particularly high absolute tumor accumulation, which is anticipated to provide an optimal signal intensity in tumor lesions during radio- and fluorescence guided surgery, thus further improving the sensitivity of the method. [^{99m}Tc]PSMA-HSG with its excellent PSMA-targeting characteristics, has great potential for providing improved surgical guidance during radio/fluorescence guided resection of prostate cancer lesions compared to currently used tracers.

1. A compound of formula (I) or a pharmaceutically acceptable salt thereof: 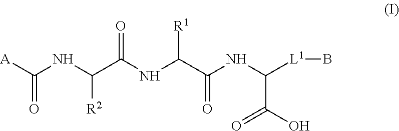
wherein
A is a group comprising a chelator group and a fluorescent dye;
R¹ is an —(C₀₋₆ alkylene)-(optionally substituted bicyclic aryl) or —(C₀₋₆ alkylene)-(optionally substituted bicyclic heteroaryl), wherein the aryl in said —(C₀₋₆ alkylene)-(optionally substituted bicyclic aryl) and the heteroaryl in said —(C₀₋₆ alkylene)-(optionally substituted bicyclic heteroaryl) are each optionally substituted with one or more groups R³;
R² is an —(C₀₋₆ alkylene)-(optionally substituted monocyclic aryl), —(C₀₋₆ alkylene)-(optionally substituted bicyclic aryl), —(C₀₋₆ alkylene)-(optionally substituted monocyclic heteroaryl), or —(C₀₋₆ alkylene)-((optionally substituted bicyclic heteroaryl), wherein the aryl in said —(C₀₋₆ alkylene)-(optionally substituted monocyclic aryl) and in said —(C₀₋₆ alkylene)-(optionally substituted bicyclic aryl) and the heteroaryl in said —(C₀₋₆ alkylene)-(optionally substituted monocyclic heteroaryl) and in said —(C₀₋₆ alkylene)-(optionally substituted bicyclic heteroaryl) are each optionally substituted with one or more groups R⁴;
each R³ is independently selected from halogen, C₁₋₆ alkyl, C₂₋₆ alkenyl, C₂₋₆ alkynyl, C₁₋₆ haloalkyl, —OR⁵, —SO₂R⁵, —NO₂, —CN, —C(O)R⁵, —C(O)OR⁵, —C(O)N(R⁵)₂, —NR⁵R⁵, —NCO, —NCS, —SR⁵, —N(R⁵)C(O)R⁵ and —N₂;
each R⁴ is independently selected from halogen, C₁₋₆ alkyl, C₂₋₆ alkenyl, C₂₋₆ alkynyl, C₁₋₆ haloalkyl, —OH, —OR⁵, —SO₂R⁵, —NO₂, —CN, —C(O)R⁵, —C(O)OR⁵, —C(O)N(R⁵)₂, NR⁵R⁵, —NCO, —NCS, —SR⁵, —NR⁵C(O)R⁵ and —N₂;
each R⁵ is independently selected from hydrogen, halogen, and C₁₋₆ alkyl;
L¹ is a linear —(C₁₋₃₀ alkylene)- group, wherein one or more —CH₂— units in said alkylene are optionally replaced by a group independently selected from —O—, —NH—, —N(C₁₋₆ alkyl)-, —CHR⁶—, —C(R⁶)₂—, —CO—, —S—, —SO—, —SO₂— or —(C₃₋₆ cycloalkylene)-; wherein each R⁶ is independently selected from halogen, C₁₋₆ alkyl, C₂₋₆ alkenyl, C₂₋₆ alkynyl, C₁₋₆ haloalkyl, —OH, —O(C₁₋₆ alkyl), —C(O)C₁₋₆ alkyl, —CN, —NH₂, —NH(C₁₋₆ alkyl) and —N(C₁₋₆ alkyl)(C₁₋₆ alkyl);
and
B is selected from —EuK and —EuE;
wherein
—EuK is 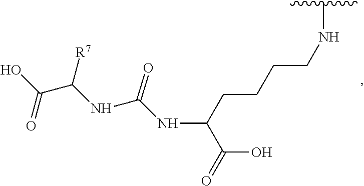
—EuE is 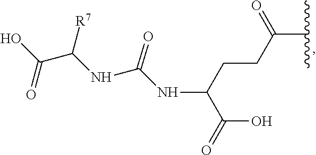
wherein R⁷ and R⁸ may be the same or different, wherein R⁷ and R⁸ are independently selected from —(C₁₋₆ alkylene)-C(O)OH, preferably wherein R⁷ and R⁸ are each —(C₂ alkylene)-C(O)OH;
wherein
the fluorescent dye is selected from Cy3, Cy3B, Cy3.5, Cy5, Cy5.5, Cy7, carbocyanine, indocarbocyanine, oxacarbocyanine, thiacarbocyanine, merocyanine, polymethine, coumarine, rhodamine, rhodamine B, xanthene, fluorescein, boron-dipyrromethane (BODIPY), VivoTag-680, Vivo Tag-S680, Vivo Tag-S750, AlexaFluor647, AlexaFluor660, AlexaFluor680, AlexaFluor700, AlexaFluor750, AlexaFluor790, Dy676, Dy677, Dy682, Dy752, Dy780, DyLight547, DyLight647, DyLight680, HiLyte Fluor 647, HiLyte Fluor 680, HiLyte Fluor 750, IRDye 800CW, IRDye 800RS, IRDye 700DX, ADS780WS, ADS830WS, ADS832WS and derivatives thereof.

2. The compound of formula (I) according to claim 1, wherein the chelator group is selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]-hexadecan (D02A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphate) (DPDP), diethylenetriaminepentaacetic acid (DTPA), ethylenediamine-N,N′-tetraacetic acid (EDTA), ethylene glycol-0,0-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridin-bis(methyl-enamine)tetraacetic acid (TMT), 1,4,7,10-tetraazacyclo-tridecan-N,N′,N″,N′″-tetraacetic acid (TRITA), triethylenetetraaminehexaacetic acid (TTHA), N,N′-bis[(6-carboxy-2-pyridyl)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl}heptanedioic acid bis-[(3-hydroxy-1,6-dimethyl-4-oxo-1,4-dihydro-pyridin-2-ylmethyl)-amide](THP), mercaptoacetyl triserine (mas₃), mercaptoacetyl triglycine (mag₃) and derivatives thereof.

3. The compound of formula (I) according to claim 1 wherein
i) B is EuK having the configuration 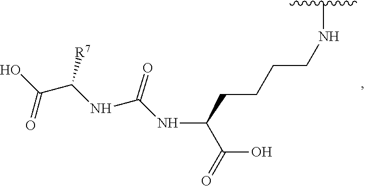
or EuE having the configuration 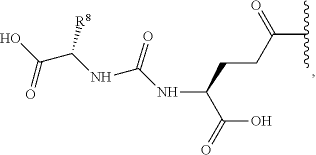
wherein R⁷ and R⁸ may be the same or different, wherein R⁷ and R⁸ are each independently selected from —(C₁₋₆ alkylene)-C(O)OH;
and/or
ii) wherein the chelator group is -mas₃ or a thioether or thioester derivative thereof, -mag₃ or a thioether or thioester derivative thereof, -DOTAGA or -DOTA, wherein -mas₃ or a thioether or thioester derivative of -mas₃ is selected from 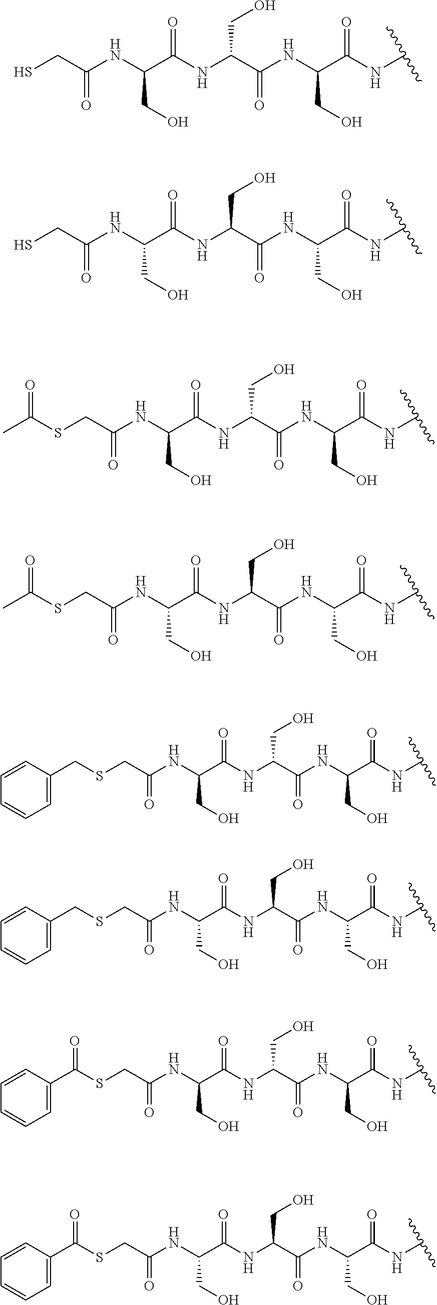
-mag₃ or a thioether or thioester derivative of -mag₃ is selected from
-DOTAGA is 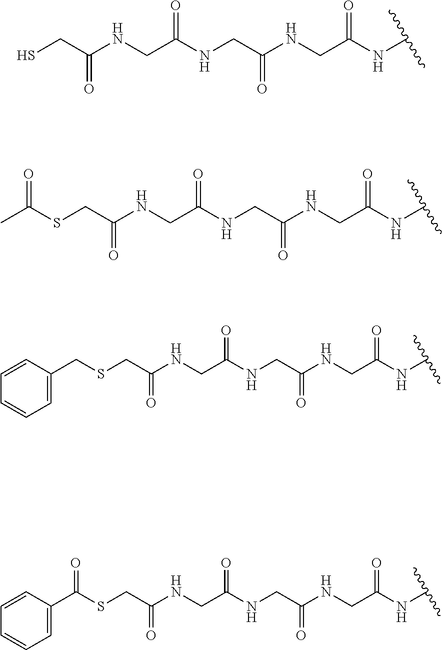 [SMILES: *NC(=O)CNC(=O)CNC(=O)CNC(=O)CS ; *NC(=O)CNC(=O)CNC(=O)CNC(=O)CSC(C)=O ; *NC(=O)CNC(=O)CNC(=O)CNC(=O)CSCc1ccccc1 ; *NC(=O)CNC(=O)CNC(=O)CNC(=O)CSC(=O)c1ccccc1] 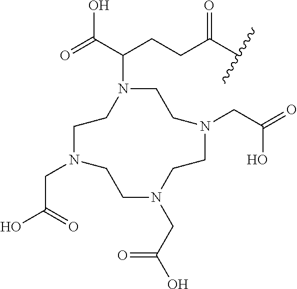 [SMILES: *C(=O)CCC(C(=O)O)N1CCN(CC(=O)O)CCN(CC(=O)O)CCN(CC(=O)O)CC1]
-DOTA is 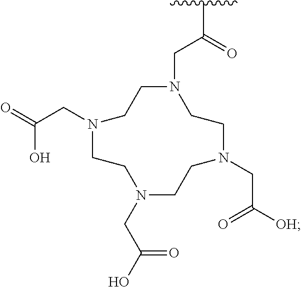 [SMILES: *C(=O)CN1CCN(CC(=O)O)CCN(CC(=O)O)CCN(CC(=O)O)CC1]
and/or
iii) wherein the fluorescent dye is Cy5, Cy 5.5, Cy7 or a derivative thereof.

4. The compound of formula (I) according to claim 3, wherein the fluorescent dye is selected from 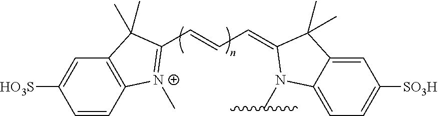
wherein
n is 2 or 3.

5. The compound of formula (I) according to claim 1, wherein R¹ is a —(C₁ alkylene)-(optionally substituted bicyclic 10-membered aryl) and R² is a —(C₁ alkylene)-(optionally substituted monocyclic 6-membered aryl), wherein the bicyclic 10-membered aryl in said —(C₁ alkylene)-(optionally substituted bicyclic 10-membered aryl) is each optionally substituted with one or more groups R³, and the monocyclic 6-membered aryl in said —(C₁ alkylene)-(optionally substituted monocyclic 6-membered aryl) is substituted with an R⁴ group —OH.

6. The compound of formula (I) according to claim 1, wherein the compound is

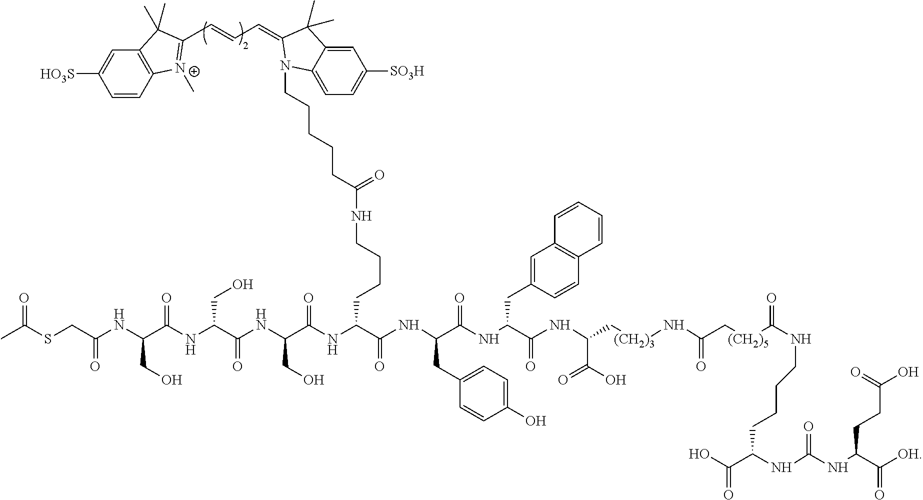

7. A composition comprising the compound of formula (I) according to claim 1 and a radionuclide.

8. The composition according to claim 7, wherein the radionuclide is ^{99m}Tc.

9. The composition according to claim 8, wherein the compound of formula (I) comprises a chelator group having at least 2 functional groups for bonding to the radionuclide ^{99m}Tc, wherein said functional group comprises an atom having 1 to 3 free electron lone pair(s), wherein said atom is O, P, N or S; and
wherein the chelator group is bidentate, tridentate or tetradentate; and/or
wherein the chelator group is cyclic or acyclic.

10. A pharmaceutical composition comprising the compound of formula (I) according to claim 1 and a pharmaceutically acceptable excipient.

11. A method for diagnosing a solid cancer in a subject, which comprises administering a compound of formula (I) according to claim 1 to said subject and imaging the solid cancer.

12. The method according to claim 11, wherein the solid cancer is selected from lung cancer, gastrointestinal cancer, colorectal cancer, colon cancer, anal cancer, liver cancer, pancreatic cancer, stomach cancer, genitourinary cancer, bladder cancer, biliary tract cancer, hepatobiliary cancer, testicular cancer, cervical cancer, ovarian cancer, uterine cancer, endometrial cancer, vaginal cancer, vulvar cancer, malignant mesothelioma, esophageal cancer, laryngeal cancer, prostate cancer, breast cancer, brain cancer, neuroblastoma, Ewing's sarcoma, osteogenic sarcoma, kidney cancer, epidermoid cancer, skin cancer, melanoma, head and/or neck cancer, mouth cancer, thymoma, Merkel-cell cancer, and neuroendocrine cancer.

13. The method according to claim 12, wherein the solid cancer is prostate cancer.

14. The method according to claim 11, wherein the imaging comprises positron emission spectroscopy (PET), single photon emission computed tomography (SPECT), scintigraphy, (intraoperative)gamma-tracing/imaging, or (intraoperative)beta-tracing; and
wherein the imaging comprises fluorescence imaging.

15. An in vitro method of imaging tissues expressing or over expressing prostrate specific membrane antigen (PSMA), the method comprising contacting said tissue with the compound of formula (I) according to claim 1.

16. The compound of formula (I) according to claim 5, having the following configuration:

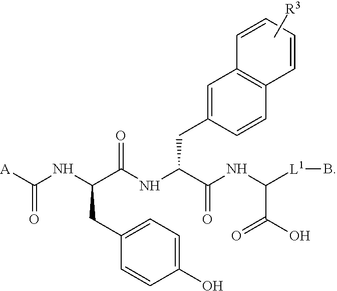

17. The composition according to claim 7, wherein the radionuclide is selected from ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁷Ga, ⁶⁸Ga, ⁸⁹Zr, ⁸⁹Y, ⁹⁰Y, ^{94m}Tc, ^{99m}Tc, ⁹⁷Ru, ¹⁰⁵Rh, ¹⁰⁹Pd, ¹¹¹Ag, ¹¹⁰In, ¹¹¹In, ¹¹³In, ¹¹⁴In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁷Lu, ¹⁸⁶Re, ¹⁸⁸Re, ¹⁹¹Pt, ¹⁹⁷Hg, ¹⁹⁸Au, ¹⁹⁹Au, ²⁰³Pb, ²¹²Pb, ²¹¹At, ²¹²Bi, ²¹³Bi, ²²³Ra, ²²⁵Ac, and ²²⁷Th, and a cationic molecule comprising ¹⁸F.

18. The composition according to claim 17, wherein said cationic molecule is ¹⁸F—[AlF]²⁺.

19. The method according to claim 14, wherein the fluorescence imaging comprises fluorescence spectroscopy.
